SEQUENCE LISTING (48 sequences)

SEQ ID NO: 1 - DNA, 2124 bp, Homo sapien
  CDS (1)...(2124)
atgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtga

SEQ ID NO: 2 - protein, 707 aa, Homo sapien
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 3 - DNA, 501 bp, Artificial Sequence
  Synthesized
atgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaa

SEQ ID NO: 4 - protein, 167 aa, Artificial Sequence
  Synthesized
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTK

SEQ ID NO: 5 - DNA, 1884 bp, Homo sapien
  CDS (1)...(1884)
atgtgtgtcccgagaagcggtttgatactgagctgcatttgcctttactgtggagttttgttgccggttctgctccctaatcttccttttctgacgtgcctgagcatgtccacattagaatctgtgacatacctacctgaaaaaggtttatattgtcagagactgccaagcagccggacacacgggggcacagaatcactgaaggggaaaaatacagaaaatatgggtttctacggcacattaaaaatgattttttacaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtga

SEQ ID NO: 6 - protein, 627 aa, Homo sapien
MCVPRSGLILSCICLYCGVLLPVLLPNLPFLTCLSMSTLESVTYLPEKGLYCQRLPSSRTHGGTESLKGKNTENMGFYGTLKMIFYKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 7 - DNA, 1659 bp, Artificial Sequence
  Synthesized
atgggtttctacggcacattaaaaatgattttttacaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaag

SEQ ID NO: 8 - protein, 553 aa, Artificial Sequence
  Synthesized
MGFYGTLKMIFYKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 9 - DNA, 261 bp, Artificial Sequence
  Synthesized
atgtgtgtcccgagaagcggtttgatactgagctgcatttgcctttactgtggagttttgttgccggttctgctccctaatcttccttttctgacgtgcctgagcatgtccacattagaatctgtgacatacctacctgaaaaaggtttatattgtcagagactgccaagcagccggacacacgggggcacagaatcactgaaggggaaaaatacagaaaatatgggtttctacggcacattaaaaatgattttttacaaa

SEQ ID NO: 10 - protein, 87 aa, Artificial Sequence
  Synthesized
MCVPRSGLILSCICLYCGVLLPVLLPNLPFLTCLSMSTLESVTYLPEKGLYCQRLPSSRTHGGTESLKGKNTENMGFYGTLKMIFYK

SEQ ID NO: 11 - DNA, 36 bp, Artificial Sequence
  Synthesized
atgggtttctacggcacattaaaaatgattttttac

SEQ ID NO: 12 - protein, 13 aa, Artificial Sequence
  Synthesized
MGFYGTLKMIFYK

SEQ ID NO: 13 - DNA, 1620 bp, Artificial Sequence
  Synthesized
atgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaag

SEQ ID NO: 14 - protein, 540 aa, Artificial Sequence
  Synthesized
MKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 15 - DNA, 147 bp, Artificial Sequence
  Synthesized
atgtcaaaaccgggaaaacctactctaaaccatggcttggttcctgttgatcttaaaagtgcaaaagagcctctaccacatcaaactgtgatgaagatatttagcattagcatcattgcccaaggcctccctttttgtcgaagacgg

SEQ ID NO: 16 - protein, 49 aa, Artificial Sequence
  Synthesized
MSKPGKPTLNHGLVPVDLKSAKEPLPHQTVMKIFSISIIAQGLPFCRRR

SEQ ID NO: 17 - DNA, 1770 bp, Homo sapien
  CDS (1)...(1770)
atgtcaaaaccgggaaaacctactctaaaccatggcttggttcctgttgatcttaaaagtgcaaaagagcctctaccacatcaaactgtgatgaagatatttagcattagcatcattgcccaaggcctccctttttgtcgaagacggatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtga

SEQ ID NO: 18 - protein, 589 aa, Homo sapien
MSKPGKPTLNHGLVPVDLKSAKEPLPHQTVMKIFSISIIAQGLPFCRRRMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 19 - protein, 41 aa, Artificial Sequence
  Synthesized
LPKELALYVLSFLEPKDLLQAAQTCRYWRILAEDNLLWREK

SEQ ID NO: 20 - protein, 281 aa, Artificial Sequence
  Synthesized
LKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWD

SEQ ID NO: 21 - protein, 13 aa, Artificial Sequence
  Synthesized
RGNPSSSQVDEEQ

SEQ ID NO: 22 - protein, 14 aa, Artificial Sequence
  Synthesized
CRGNPSSSQVDEEQ

SEQ ID NO: 23 - protein, 18 aa, Artificial Sequence
  Synthesized
CQRLPSSRTHGGTESLKG

SEQ ID NO: 24 - protein, 16 aa, Artificial Sequence
  Synthesized
PVDLKSAKEPLPHQTV

SEQ ID NO: 25 - protein, 17 aa, Artificial Sequence
  Synthesized
CPVDLKSAKEPLPHQTV

SEQ ID NO: 26 - protein, 14 aa, Artificial Sequence
  Synthesized
KRKLDHGSEVRSFS

SEQ ID NO: 27 - protein, 15 aa, Artificial Sequence
  Synthesized
CKRKLDHGSEVRSFS

SEQ ID NO: 28 - RNA, 501 nt, Artificial Sequence
  Synthesized
augaaucaggaacugcucucugugggcagcaaaagacgacgaacuggaggcucucugagagguaacccuuccucaagccagguagaugaagaacagaugaaucgugugguagaggaggaacagcaacagcaacucagacaacaagaggaggagcacacugcaaggaauggugaaguuguuggaguagaaccuagaccuggaggccaaaaugauucccagcaaggacaguuggaagaaaacaauaauagauuuauuucgguagaugaggacuccucaggaaaccaagaagaacaagaggaagaugaagaacaugcuggugaacaagaugaggaggaugaggaggaggaggagauggaccaggagagugacgauuuugaucagucugaugauaguagcagagaagaugaacauacacauacuaacagugucacgaacuccaguaguauuguggaccugcccguucaccaacucuccuccccauucuauacaaaaacaacaaaa

SEQ ID NO: 29 - RNA, 261 nt, Artificial Sequence
  Synthesized
augugugucccgagaagcgguuugauacugagcugcauuugccuuuacuguggaguuuuguugccgguucugcucccuaaucuuccuuuucugacgugccugagcauguccacauuagaaucugugacauaccuaccugaaaaagguuuauauugucagagacugccaagcagccggacacacgggggcacagaaucacugaaggggaaaaauacagaaaauauggguuucuacggcacauuaaaaaugauuuuuuacaaa

SEQ ID NO: 30 - RNA, 147 nt, Artificial Sequence
  Synthesized
augucaaaaccgggaaaaccuacucuaaaccauggcuugguuccuguugaucuuaaaagugcaaaagagccucuaccacaucaaacugugaugaagauauuuagcauuagcaucauugcccaaggccucccuuuuugucgaagacgg

SEQ ID NO: 31 - protein, 410 aa, Homo sapien
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 32 - protein, 395 aa, Homo sapien
MKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 33 - protein, 15 aa, Artificial Sequence
  Synthesized
MPRERRERDAKERDT

SEQ ID NO: 34 - DNA, 2063 bp, Artificial Sequence
  Synthesized
agtcaaggctttgacagggcatagtctcctccaataatcttctccgttctctctcattattccctcgagttcttctcagtcaagctgcatgtatgtatgtgtgtcccgagaagcggtttgatactgagctgcatttgcctttactgtggagttttgttgccggttctgctccctaatcttccttttctgacgtgcctgagcatgtccacattagaatctgtgacatacctacctgaaaaaggtttatattgtcagagactgccaagcagccggacacacgggggcacagaatcactgaaggggaaaaatacagaaaatatgggtttctacggcacattaaaaatgattttttacaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaaaaaaaaaa

SEQ ID NO: 35 - DNA, 2255 bp, Artificial Sequence
  Synthesized
aatgtgaaaacctttgcatcttctgatagtctagccaaggtccaagaagtagcaagctggcttttggaaatgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgc

SEQ ID NO: 36 - protein, 15 aa, Artificial Sequence
  Synthesized
CSLIPTPDKEDDDRV

SEQ ID NO: 37 - protein, 15 aa, Artificial Sequence
  Synthesized
CSLIPTPDKEDDDRV

SEQ ID NO: 38 - protein, 12 aa, Artificial Sequence
  Synthesized
SGLLTPPQSGKK

SEQ ID NO: 39 - protein, 12 aa, Artificial Sequence
  Synthesized
SGLLTPPQSGKK

SEQ ID NO: 40 - protein, 13 aa, Artificial Sequence
  Synthesized
CSGLLTPPQSGKK

SEQ ID NO: 41 - protein, 13 aa, Artificial Sequence
  Synthesized
CSGLLTPPQSGKK

SEQ ID NO: 42 - DNA, 28 bp, Artificial Sequence
  Synthesized
gcaagcttatgggtttctacggcacatt

SEQ ID NO: 43 - DNA, 27 bp, Artificial Sequence
  Synthesized
atgggccctgctcttcacttcatgtcc

SEQ ID NO: 44 - DNA, 21 bp, Artificial Sequence
  Synthesized
cttttggaaatgaatcaggaa

SEQ ID NO: 45 - DNA, 21 bp, Artificial Sequence
  Synthesized
catgtatgtatgtgtgtcccg

SEQ ID NO: 46 - protein, 395 aa, Artificial Sequence
  Synthesized
MKEDGGAEFSARSRKRKANVTVFLQDPDEEMAKIDRTARDQCGSQPWDNNAVCADPCSLIPXPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 47 - protein, 395 aa, Artificial Sequence
  Synthesized
MKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLXPPQSGKKQSSGPEMA

SEQ ID NO: 48 - protein, 395 aa, Artificial Sequence
  Synthesized
MKEDGGAEFSARSRKRKANVTVFLQDPDEEMAKIDRTARDQCGSQPWDNNAVCADPCSLIPXPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLXPPQSGKKQSSGPEMA

(19) United States
(12) Patent Application Publication
(10) Pub. No.: US 2003/0143582 A1
(43) Pub. Date: Jul. 31, 2003

(54) Human CDC4 modulates cyclin E degradation

(75) Inventors: Steve Reed, Cardiff, CA (US); Charles Spruck, San Diego, CA (US); Heimo Strohmaier, San Diego, CA (US); Olle Sangfelt, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/245,618

(22) Filed: Sep. 16, 2002

Related U.S. Application Data
(60) Provisional application No. 60/322,947, filed on Sep. 14, 2001.
(60) Provisional application No. 60/404,116, filed on Aug. 15, 2002.

(51) Int. Cl. C12Q 1/68; G01N 33/53; C12N 9/16; C12P 21/02; C12N 5/06; C07K 16/40; C07H 21/04

(52) U.S. Cl. 435/6; 435/69.1; 435/196; 435/320.1; 435/325; 530/388.26; 435/338; 435/7.1; 536/23.2

(57) ABSTRACT

The present invention provides compositions including polypeptides useful for regulating the cell cycle, proliferation, tumorigenesis, and genomic stability, mutants of such polypeptides useful in identifying abnormal cells and diagnosing disease, antibodies useful for identifying the polypeptides, and polynucleotides that encode the polypeptides.

### GOVERNMENT SUPPORT CLAUSE

This invention was made with government support under contract CA78343 awarded by National Cancer Institute. The United States Government has certain rights to the invention.

### BACKGROUND OF THE INVENTION

1. Field of the Invention

The present invention is related to the field of cell cycle regulation and cell proliferation. In particular, this invention is related to modulation of cyclin E ubiquitination and proteolysis, hCdc4, and compositions and methods associated therewith.

2. State of the Art

A transient accumulation of cyclin E at the G1/S boundary of the cell cycle is critical for the initiation of DNA replication and other S phase functions (Reed (1996) Biochim Biophys Acta 1287:151-153; Reed (1997) Cancer Surv 29:7-23; and Sauer et al. (1995) Prog Cell Cycle Res 1:125-39). The profile of cyclin E expression at the G1/S boundary is achieved by periodic transcription coupled with cyclin E specific ubiquitin-mediated proteolysis (Won et al. (1996) EMBO J 15:4182-93).

Understanding the factors and regulatory events that modulate cyclin E expression is important because constitutive expression of cyclin E leads to genomic instability and elevated cyclin E protein levels are associated with a variety of malignancies (Donnellan et al. (1999) Faseb J 13:773-780; Sandhu et al. (2000) Cancer Detect Prev 24:107-118; and Spruck et al. (1999) Nature 401:297-300).

### SUMMARY OF THE INVENTION

The present inventors discovered that cyclin E degradation is modulated by phosphorylation of cyclin E residues threonine-62 and threonine-380 (T62-phospho and T380-phospho, respectively). The inventors further discovered a polypeptide, referred to herein as human Cdc4 (hCdc4), including α and β isoforms thereof, that couples phosphorylated cyclin E with an SCF ubiquitin ligase complex and modulates degradation of cyclin E in the early S phase of the cell cycle. The inventors determined that a cellular accumulation of phosphorylated cyclin E, or increased ratio of phosphorylated cyclin E to non-phosphorylated cyclin E, is predictive of the tumorigenic phenotype, independent of total cyclin E levels. The inventors identified defects in the SCF^{hCdC4} (a complex of SCF and hCdc4) pathway of cyclin E degradation that result in a cellular accumulation of phosphorylated cyclin E and disclose that elevated phosphorylated cyclin E comprises a tumorigenic phenotype.

One embodiment of the present invention provides a tumor marker comprising cellular accumulation of a phosphorylated cyclin E. In one example, the phospho-cyclin E comprises a T380-phospho cyclin E (cyclin E phosphorylated at threonine 380). Preferably, the phosphorylated cyclin E tumor marker comprises a T62-phospho cyclin E (cyclin E phosphorylated at threonine 62) and more preferably a cyclin E including a T62-phosphorylation and a T380-phosphorylation. Another embodiment provides a method of detecting a tumor in a subject comprising identifying an accumulation of a phosphorylated cyclin E in cells of the subject. In one embodiment the tumor comprises an endometrial tumor. In other embodiments, the phospho-cyclin E comprises a marker of a lung tumor, a head or neck tumor, a testicular tumor, or a breast tumor. In an alternative embodiment, the phospho-cyclin E comprises a marker for a neoplasia.

Certain embodiments provide a method of detecting a tumor, comprising: detecting an accumulation of a phosphorylated cyclin E in a sample suspected of including a tumorigenic phenotype (e.g., a sample of cells; a tissue; or a fluid, such as blood, lymph, or saliva). A preferred phosphorylated cyclin E includes a T62-phosphorylation, and more preferably, further includes a T380-phosphorylation.

One embodiment of the present invention provides that genetic or expressed mutations in an α-hCdc4 (optionally, a β-hCdc4) correlate with an accumulation of phosphorylated cyclin E. Another embodiment provides that a genetic or an expressed mutation in an α-hCdc4 is useful, for example, as a tumor marker, preferably an endometrial tumor marker.

Additional embodiments include: 1) polypeptides and polynucleotides of hCdc4 isoforms and variants, mutants, immunogens, and antibodies associated therewith; 2) certain polypeptides and polynucleotides of cyclin E and mutants, immunogens, and antibodies associated therewith; 3) vectors and host cells capable of expressing hCdc4 and cyclin E compositions of the present invention; 4) methods use thereof; including methods of detecting the polypeptides and polynucleotides, methods detecting a neoplasm and tumor, methods of detecting changes in expression; 5) diagnostic assays; and 6) kits.

### BRIEF DESCRIPTION OF THE DRAWINGS

FIG. 1A provides diagrams (upper and lower panels) that depict an example of a genomic structure for the human CDC4 (hCDC4) gene (upper panel) and messenger RNA (mRNA) expression products (lower panel). The gene locus maps to human chromosome region 4q32. The gene includes 4 untranslated and 13 coding exons spanning approximately 210 kilobases (kb) of the human genome. The 4 untranslated exons are not shown. Expressed isoforms of hCdc4 are α-hCdc4, β-hCdc4, and γ-hCdc4 which vary by the first exons and include a common C-terminal domain. The first exon of each hCDC4 isoform is referred to herein as α-exon 1, β-exon 1, and γ-exon 1; respectively.

The diagram in the lower panel of FIG. 1A depicts each exon 1 alternatively spliced to common exons 2-11, thereby forming the α-, β-, and γ-hCdc4 isoforms.

FIG. 1B is a diagram that depicts embodiments of hCdc4 polypeptide isoforms α-hCdc4, β-hCdc4, and γ-hCdc4 (not to scale). The diagram shows examples of sequence identifiers for each substantially full length hCdc4 polypeptide isoform, the C-terminal common region, and the alternatively spliced N-terminal exons. Inclusion of these sequence identifiers is not meant to limit the depicted embodiment, but to aid in more easily understanding specific embodiments disclosed herein. The vertical lines on the hCdc4 isoforms are meant to depict that the isoforms have a common C-terminal region, but unique N-terminal regions.

### DETAILED DESCRIPTION OF THE INVENTION

The present invention provides compositions including polynucleotide and polypeptide useful for regulating the cell cycle, proliferation, tumorigenesis, and genomic stability of cells. This invention also provides mutants of such polynucleotides and polypeptides and assays useful in identifying abnormal cells and diagnosing disease. This inventor further provides antibodies useful for identifying polypeptides of the present invention and useful for diagnostic assays. These and other embodiments of the invention are disclosed in detail herein.

The present inventors discovered that the yeast F-box protein, Cdc4, specifically couples the yeast SCF complex to cyclin E resulting in ubiquitination and proteolysis of the cyclin E. The inventors also discovered a human protein that specifically couples a SCF type complex to the ubiquitination and degradation of cyclin E in human cells. The human coupling protein is referred to herein as human Cdc4 (hCdc4). A complex of hCdc4 and human SCF is referred to herein as SCF^{hCdc4}.

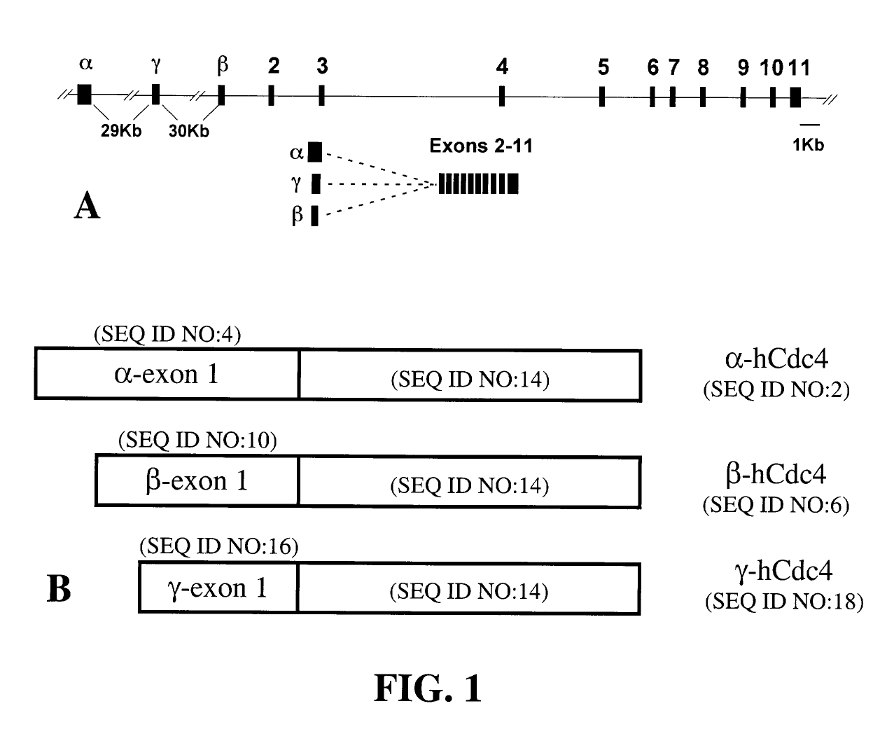

The present inventors discovered two hCdc4 isoforms, referred to herein as α-hCdc4 and β-hCdc4 (see, FIGS. 1A and 1B); identified the hCdc4 gene; and determined the genomic structure of the hCDC4 gene (see, FIG. 1A). A third hCdc4 isoform is referred to herein as γ-hCdc4. Additional hCdc4 isoforms are contemplated. Alternatively, the α-hCdc4, β-hCdc4, and γ-hCdc4 isoforms of hCdc4 are herein referred to as: hCdc4 isoform 1, hCdc4 isoform 2, and hCdc4 isoform 3; respectively.

Referring to FIG. 1A, in one embodiment, the three hCdc4 isoforms arise from alternative splicing of ten common 3′ exons to three different 5′ coding exons (α, β, and γ forms of exon 1). The C-terminal common region of hCdc4 includes an F-box domain (e.g., SEQ ID NO:19) and a WD40 repeat region having approximately seven or eight WD40 repeats (e.g., SEQ ID NO:20).

Certain embodiments of the present invention provide the following (a) turnover of human cyclin E depends on hCdc4 mediated coupling of cyclin E to a SCF type protein ubiquitin ligase, (b) hCdc4 function is rate-limiting for determining cyclin E degradation and protein levels, (c) overexpression of hCdc4 in human cells reduces the steady state level of cyclin E, (d) human cells that include expression of a dominant negative form of hCdc4 (e.g., deletion of the F-box region) have increased cyclin E levels, (e) the gene encoding hCdc4 is mutated in cell lines derived from cancers including breast and endometrial cancers, (f) hCdc4 is mutated in at least 16% of human endometrial tumors, (g) certain mutations of the α-exon 1 region of the α-hCdc4 isoform and of the β-exon 1 region of the β-hCdc4 isoform are observed in human endometrial tumors, (h) mutations in the C-terminal common region of hCdc4 lead to a specific increase in phosphorylated cyclin E, (i) levels of phosphorylated cyclin E are specifically associated with human tumors and tumor aggression (e.g., predicted outcome), and (j) the hCDC4 gene locus is at human chromosome 4q32 which is deleted in over 30% of a wide variety of human tumors.

It is one embodiment of the present invention that the hCDC4 gene is mutated in primary human tumors. It is another embodiment of the present invention that hCdc4 expression is aberrant in primary human tumors (e.g., decreased expression of hCdc4 mRNA and/or appearance of an hCdc4 mRNA of aberrant length).

Certain embodiments provide that α-hCdc4 is expressed in substantially all tissues and is the predominately expressed isoform of hCdc4, except in skeletal muscle and brain, wherein the β-hCdc4 and γ-hCdc4 isoforms are the predominately expressed isoforms. Cells of the skeletal muscle and brain are among the slowest proliferating cells during the majority of adult human life and include little cyclin E expression.

It is a preferred embodiment of the present invention that α-hCdc4 expression is aberrant in primary human tumors (including aberrant expression arising from genetic mutations or otherwise). One embodiment provides a method of detecting a tumor cell comprising detecting a deficiency in an α-hCdc4 in the cell or a sample of tumor cells. Certain embodiments provide detecting a defect in the α-hCdc4 gene, an α-hCdc4 RNA (preferably mRNA), or an α-hCdc4 polypeptide. A defect in an α-hCdc4 gene is detected in one embodiment using single strand conformation polymorphism (SSCP). For example, using polymerase chain reaction (PCR) amplification of α-hCdc4 exon sequences (primers are generally selected in the intron sequence). A defect in an α-hCdc4 mRNA is detected in one example using SSCP screening from RT-PCR amplification of α-hCdc4 mRNA. In another example, defects in α-hCcd4 mRNA and/or polypeptide can be assayed by determining cellular α-hCdc4 mRNA and/or α-hCdc4 polypeptide content (e.g., using a complementary nucleic acid sequence or a specific antibody, preferably labeled with a detectable marker).

In one embodiment, a functional α-hCdc4 enhances cyclin E ubiquitination. An assay of cyclin E ubiquitination is set forth herein. In another embodiment, a functional α-hCdc4 binds a phosphorylated cyclin E, preferentially a cyclin E including phosphorylation of the T62 and T380 residues (optionally, the T62 residue). One embodiment provides a method of decreasing a cyclin E content of a cell comprising administering an α-hCdc4 (optionally a β-hCdc4) to the cell. The α-hCdc4 can be administered using common methods known in the art for introducing polypeptides and polynucleotides capable of expressing such polypeptides to cells. For example, electroporation, calcium phosphate precipitation, inclusion in a liposome or lipid useful for transfection, and viral transduction (e.g., using an adenovirus or retrovirus vector tailored to transduction of a nucleic acid insert. Targeting mechanisms include incorporation of cell specific molecules, preferably receptor ligands. A particularly preferred targeting receptor ligand comprises an integrin or integrin receptor binding ligand. One embodiment provides for administration of an α-hCdc4 (polypeptide or polynucleotide) to a group of cells, preferably a tumor, having an elevated level of phospho-cyclin E; thereby reducing the level of phospho-cyclin E in the cells. In such embodiment, the α-hCdc4 (optionally, including a transfer agent) is administered by injecting a composition including the α-hCdc4 in a pharmaceutically acceptable excipient. Additional methods of administration are provided in U.S. Pat. No. 6,312,956 to Lane and U.S. Pat. No. 5,807,746 to Lin et al., each patent incorporated herein by reference in its entirety. It is preferred that the level of phospho-cyclin E is detected after the administration of the α-hCdc4, thereby providing a comparison value for determining effectiveness of the administration of the hCdc4 to the reduction in phospho-cyclin E.

Polypeptides

α-hCdc4 Polypeptides

One embodiment of the present invention provides a purified polypeptide comprising an amino acid sequence including SEQ ID NO:4 (see, FIG. 1B). In one embodiment, the instant amino acid sequence further includes SEQ ID NO:14, wherein SEQ ID NO:4 is operably linked to SEQ ID NO:14 (preferably the C-terminus of SEQ ID NO:4 is linked to the N-terminus of SEQ ID NO:14, see, FIG. 1B). An exemplary polypeptide including SEQ ID NO:4 operably linked to SEQ ID NO:14 comprises the amino acid sequence set forth in SEQ ID NO:2. Thus, another embodiment of the present invention provides a purified polypeptide comprising the amino acid set forth in SEQ ID NO:2. An alternative embodiment provides a polypeptide comprising an amino acid sequence including SEQ ID NO:4, but not including SEQ ID NO:14. In one embodiment, an α-exon 1 of an α-hCdc4 polypeptide comprises SEQ ID NO:4. In another embodiment, an α-hCdc4 polypeptide includes SEQ ID NO:2 (see, FIG. 1B).

One embodiment provides a composition comprising a purified polypeptide including an amino acid sequence set forth in SEQ ID NO:4 (optionally, an α-exon 1 sequence, see FIG. 1B). In another embodiment, the composition comprises a polypeptide consisting essentially of the amino acid sequence set forth in SEQ ID NO:4 (optionally, an α-exon 1 sequence). In another embodiment, the composition comprises a purified polypeptide including the amino acid sequence set forth in SEQ ID NO:2 (optionally, an α-hCdc4 sequence). In a further embodiment, the instant composition comprises a purified polypeptide consisting essentially of the amino acid sequence set forth in SEQ ID NO:2 (optionally, an α-hCdc4 sequence).

One embodiment of the present invention provides a purified, substantially full length α-hCdc4 polypeptide. Characteristic embodiments of a substantially full length α-hCdc4 polypeptide include, but are not limited to, one or more of: (a) a polymer of approximately 707 amino acids; (b) an approximate molecular weight determined by gel electrophoresis (native and non-native) of 110 kilo Dalton (kDa); (c) a calculated molecular weight of approximately 79,613 Dalton based on SEQ ID NO:2; (d) a sequence substantially similar or identical to SEQ ID NO:2; (e) an N-terminal portion (the α-exon 1 portion) that is highly acidic; (f) and an N-terminal portion (the α-exon 1 portion) having a net negative charge (which is contemplated to affect the aberrant migration of α-hCdc4 on polyacrylamide gels compared to gel electrophoresis).

One embodiment provides a immunogenic composition comprising a purified polypeptide consisting essentially of 6 to 167 consecutive amino acid residues of SEQ ID NO:4. In certain embodiments, the immunogenic composition comprises a polypeptide consisting essentially of 10 to 20 consecutive amino acid residues of SEQ ID NO:4. Optionally the polypeptide consists essentially of 10 to 167 consecutive amino acid residues of SEQ ID NO:4. In general, and as used herein, 6 amino acids is considered the minimal length of a polypeptide capable of being specifically recognized by an antibody. Typically a polypeptide 10-mer to 20-mer, or even a 40-mer are nearly certain to be specifically recognized by an antibody (the “-mer” designation refers to the length of the polypeptide in amino acid residues). Accordingly, polypeptides of 6, 7, 8, 9, 10, 11, 12, 13, 14, 15, 16, 17, 18, 19, or 20 consecutive amino acids are ideally suited for use in an α-exon 1 or an α-hCdc4 immunogenic composition.

In another preferred embodiment, the polypeptide is combined with an immunogenicity enhancing agent. Immunogenicity enhancing agents and their combination with polypeptides are well known in the art. For example, one embodiment provides an α-exon 1 immunogenic polypeptide combined with an adjuvant, such as Freund's complete adjuvant, or a carrier particle, such as keyhole limpet hemocyanin (KLH) or colloidal metals (e.g., colloidal gold) for enhancing an immunogenic response to the α-exon 1 polypeptide.

In a preferred embodiment, the immunogenic polypeptide consists essentially of RGNPSSSQVDEEQ (SEQ ID NO:21). In certain embodiments it is desirable to combine a linking agent with the polypeptide. For example, in one preferred embodiment, the immunogenic composition includes an N-terminal cysteine residue linked to the polypeptide (when the polypeptide does not already include a cysteine residue). The cysteine residue is useful for linking the immunogenic polypeptide to a carrier, such as KLH. Accordingly, in one embodiment, the immunogenic composition comprises a cysteine operably linked to SEQ ID NO:21, which is alternatively referred to herein as SEQ ID NO:22 (CRGNPSSSQVDEEQ).

The present embodiments of a composition comprising an immunogen of α-exon 1 are useful, for example, for raising an antibody that specifically recognizes an α-hCdc4 polypeptide and in performing diagnostic assays to detect, identify or measure α-hCdc4 polypeptide in a sample (e.g., as a control or standard). An α-hCdc4 polypeptide can now be distinguished from the β-hCdc4 and/or γ-hCdc4 isoforms in a sample. Examples of a sample include, but are not limited to: a cell, tissue, bodily fluid, laboratory specimen, and the like. Measurements can be made in vitro, in vivo, and/or ex vivo; including from partially extracted and/or purified extracts thereof.

β-hCdc4 Polypeptides

One embodiment of the present invention provides a purified polypeptide comprising an amino acid sequence including SEQ ID NO:10 (see, FIG. 1B). In one embodiment, the instant amino acid sequence further includes SEQ ID NO:14, wherein SEQ ID NO:10 is operably linked to SEQ ID NO:14 (preferably the C-terminus of SEQ ID NO:10 is linked to the N-terminus of SEQ ID NO:14, see, FIG. 1B). An exemplary polypeptide including SEQ ID NO:10 operably linked to SEQ ID NO:14 comprises the amino acid sequence set forth in SEQ ID NO:6. Thus, another embodiment of the present invention provides a purified polypeptide comprising the amino acid set forth in SEQ ID NO:6. An alternative embodiment provides a polypeptide comprising an amino acid sequence including SEQ ID NO:10, but not including SEQ ID NO:14. In one embodiment, a β-exon 1 of a -hCdc4 polypeptide comprises SEQ ID NO:10. In another embodiment, a β-hCdc4 polypeptide includes SEQ ID NO:6 (see, FIG. 1B).

One embodiment provides a composition comprising a purified polypeptide including an amino acid sequence set forth in SEQ ID NO:10 (optionally, a β-exon 1 sequence, see FIG. 1B). In another embodiment, the composition comprises a polypeptide consisting essentially of the amino acid sequence set forth in SEQ ID NO:10 (optionally, a β-exon 1 sequence). In another embodiment, the composition comprises a purified polypeptide including the amino acid sequence set forth in SEQ ID NO:6 (optionally, a β-hCdc4 sequence). In a further embodiment, the instant composition comprises a purified polypeptide consisting essentially of the amino acid sequence set forth in SEQ ID NO:6 (optionally, a β-hCdc4 sequence).

One embodiment of the present invention provides a purified, substantially full length β-hCdc4 polypeptide. Characteristic embodiments of a substantially full length β-hCdc4 polypeptide include, but are not limited to, one or more of: (a) a polymer of approximately 627 amino acids; (b) an approximate molecular weight determined by gel electrophoresis of 69 kDa; (c) a calculated molecular weight of approximately 70,278 Dalton based on SEQ ID NO:6; (d) and a sequence substantially similar or identical to SEQ ID NO:6.

One embodiment provides a immunogenic composition comprising a purified polypeptide consisting essentially of 6 to 87 consecutive amino acid residues of SEQ ID NO:10. In certain embodiments, the immunogenic composition comprises a polypeptide consisting essentially of 10 to 20 (optionally; 10 to 87 or 7, 8, 9, 10, 11, 12, 13, 14, 15, 16, 17, 18, 19, or 40 consecutive amino acid residues of SEQ ID NO:10.

In another preferred embodiment, the polypeptide is combined with an immunogenicity enhancing agent. For example, one embodiment provides a β-exon 1 immunogenic polypeptide combined with an adjuvant, such as Freund's complete adjuvant, or a carrier particle, such as keyhole limpet hemocyanin (KLH) or colloidal metals (e.g., colloidal gold) for enhancing an immunogenic response to the β-exon 1 polypeptide.

In a preferred embodiment, the immunogenic polypeptide consists essentially of CQRLPSSRTHGGTESLKG (SEQ ID NO:23). In the instant embodiment, the immunogenic fragment of the β-exon 1 includes an N-terminal cysteine residue useful for linking to an immunogenicity enhancing agent. However, including another linker is contemplated.

The present embodiments of a composition comprising an immunogen of β-exon 1 are useful, for example, to raise an antibody that specifically recognizes a β-hCdc4 polypeptide and in performing diagnostic assays to detect, identify or measure β-hCdc4 polypeptide in a sample (e.g., as a control or standard). A β-hCdc4 polypeptide can now be distinguished from the α-hCdc4 and/or γ-hCdc4 isoforms in a sample.

γ-exon 1 Specific Immunogens

One embodiment provides a immunogenic composition comprising a purified polypeptide consisting essentially of 6 to 49 consecutive amino acid residues of SEQ ID NO:16. In preferred embodiments, the immunogenic composition comprises a polypeptide consisting essentially of 6 to 20 consecutive amino acid residues of SEQ ID NO:16. Optionally, the polypeptide consists essentially of 10 to 18 consecutive amino acid residues of SEQ ID NO:16. In a preferred embodiment, the polypeptide consists essentially of 9 to 15 consecutive amino acid residues of SEQ ID NO:16.

In another preferred embodiment, the polypeptide is combined with an immunogenicity enhancing agent. For example, one embodiment provides a γ-exon 1 immunogenic polypeptide combined with an adjuvant, such as Freund's complete adjuvant, or a carrier particle, such as keyhole limpet hemocyanin (KLH) or colloidal metals (e.g., colloidal gold) for enhancing an immunogenic response to the γ-exon 1 polypeptide.

In a preferred embodiment, the immunogenic polypeptide consists essentially of PVDLKSAKEPLPHQTV (SEQ ID NO:24). In another preferred embodiment, the composition includes a cysteine residue linked to the N-terminus of SEQ ID NO:24 (forming a composition including CPVDLKSAKEPLPHQTV (SEQ ID NO:25) which is useful for linking the polypeptide to a carrier, such as KLH).

The present embodiments of a composition comprising an immunogen of γ-exon 1 are useful, for example, to raise an antibody that specifically recognizes a γ-hCdc4 polypeptide and in performing diagnostic assays to detect, identify or measure γ-hCdc4 polypeptide in a sample (e.g., as a control or standard). A γ-hCdc4 polypeptide can now be distinguished from the α-hCdc4 and/or β-hCdc4 isoforms in a sample.

hCdc4 C-Terminal Polypeptide and Immunogens Thereof

One embodiment of the present invention provides a purified polypeptide consisting essentially of a SEQ ID NO:14, or an immunogenic fragment thereof. In one embodiment, the immunogenic fragment of SEQ ID NO:14 is KRKLDHGSEVRSFS (SEQ ID NO:26)

One embodiment provides a purified immunogenic composition comprising a polypeptide consisting essentially of 6 to 20 consecutive amino acid residues of SEQ ID NO:14. In an alternative embodiment, the polypeptide consists essentially of 10 to 18 (optionally, 11 to 15) consecutive amino acid residues. In the present embodiment, it is preferred that the polypeptide does not include an amino acid sequence set forth in SEQ ID NO:4 (e.g., α-exon 1), SEQ ID NO:10 (e.g., β-exon 1), or SEQ ID NO:16 (e.g., γ-exon 1).

Cyclin E

One embodiment of the present invention provides a purified polypeptide comprising a cyclin E polypeptide wherein the polypeptide includes a phosphorylated T62 (threonine-62) residue (alternatively written, a T62-phospho or a T62-phosphorylation). In the present embodiment, the polypeptide is substantially free of contaminants including a cyclin E that does not include the T62-phosphorylation, polyacrylamide, and kinase polypeptides. In another embodiment, the purified polypeptide comprising the T62-phospho further includes a phosphorylation at T380.

As used herein, the nomenclature for the T62 site (residue) of a cyclin E is derived from cyclin E isoform 2 (SEQ ID NO:32). Cyclin E isoform 2 was identified first, historically. Residue numbers 62 and 380 of cyclin E isoform 2 (SEQ ID NO:32) are each threonine amino acid residues. It was later determined, historically, that cyclin E has a 15 amino acid N-terminal portion (SEQ ID NO:33) that was deleted in cyclin E isoform 2. This cyclin E, with the 15 amino acid N-terminal sequence is referred to in the art as cyclin E isoform 1 (SEQ ID NO:31). As used herein, a reference to T62 of cyclin E includes the meaning of: (a) the threonine at position 62 of SEQ ID NO:32, and (b) the threonine at position 77 (62+15) of SEQ ID NO:31. As used herein, a reference to T380 of cyclin E includes the meaning of: (a) the threonine at position 380 of SEQ ID NO:32, and (b) the threonine at position 395 (380+15) of SEQ ID NO:31. In another embodiment, the T62 of cyclin E is the threonine residue at position six of CSLIPTPDKEDDDRV (SEQ ID NO:37). In another embodiment, the T380 of cyclin E is the threonine residue at position five of SGLLTPPQSGKK (SEQ ID NO:39). See FIG. 2 for a diagram depicting an alignment of cyclin E isoforms 1 and 2.

One embodiment provides a purified polypeptide comprising an amino acid sequence CSLIPTPDKEDDDRV (SEQ ID NO:36), wherein the threonine residue at position six includes a phosphorylation. In another embodiment, the purified consists essentially of the amino acid sequence CSLIPTPDKEDDDRV (SEQ ID NO:36), wherein the threonine residue at position six includes a phosphorylation (T62-phospho).

One embodiment provides a purified polypeptide comprising an amino acid sequence CSLIPTPDKEDDDRV (SEQ ID NO:37), wherein the threonine residue at position six does not include a phosphorylation. The present purified polypeptide is substantially free of contaminants including a phosphorylated cyclin E having a phosphorylation at T62, polyacrylamide, and a kinase. In another embodiment, the purified polypeptide consists essentially of the amino acid sequence CSLIPTPDKEDDDRV (SEQ ID NO:37), wherein the threonine residue at position six does not include a phosphorylation.

One embodiment provides a purified polypeptide comprising an amino acid sequence SGLLTPPQSGKK (SEQ ID NO:38), wherein the threonine residue at position five includes a phosphorylation. The present purified polypeptide is substantially free of contaminants including a phosphorylated cyclin E that does not have a phosphorylation at T380, polyacrylamide, and a kinase. In another embodiment, the purified polypeptide consists essentially of the amino acid sequence SGLLTPPQSGKK (SEQ ID NO:38), wherein the threonine residue at position five includes a phosphorylation.

One embodiment provides a purified polypeptide comprising an amino acid sequence SGLLTPPQSGKK (SEQ ID NO:39), wherein the threonine residue at position five does not include a phosphorylation. Again, the purified polyeptide is substantially free of contaminants including a cyclin E having a T380-phosphorylation. In another embodiment, the polypeptide consists essentially of the amino acid sequence SGLLTPPQSGKK (SEQ ID NO:39), wherein the threonine residue at position five does not include a phosphorylation.

One embodiment provides a purified polypeptide comprising an amino acid sequence CSGLLTPPQSGKK (SEQ ID NO:40), wherein the threonine residue at position six includes a phosphorylation. Alternatively, the polypeptide consists essentially of the amino acid sequence CSGLLTPPQSGKK (SEQ ID NO:40), wherein the threonine residue at position six includes a phosphorylation.

One embodiment provides a purified polypeptide comprising an amino acid sequence CSGLLTPPQSGKK (SEQ ID NO:41), wherein the threonine residue at position six does not include a phosphorylation. Alternatively, the polypeptide consists essentially of the amino acid sequence CSGLLTPPQSGKK (SEQ ID NO:41), wherein the threonine residue at position six does not include a phosphorylation.

Contamination of a cyclin E sample referring to including/not including a phospho-T62/phospho-T380 can be removed, for example, using an affinity matrix with one or more antibody specific to phospho-T62 cyclin E, phospho-T380 cyclin E, non-phospho-T62 cyclin E, and non-phospho-T380 cyclin E.

In certain embodiments, wherein a polypeptide is used to raise an antibody to the polypeptide, a cysteine residue is added to the polypeptide as a linker for enhancing the immunogenicity of the polypeptide when combined with a carrier, operably linked by the linker.

Variants

One embodiment of the present invention provides a polypeptide, comprising (optionally, consisting essentially of) an amino acid sequence that is 95% or more identical to an α-hCdc4 polypeptide set forth in SEQ ID NO:2 and includes an activity substantially similar to SEQ ID NO:2. Examples of the activity substantially similar to SEQ ID NO:2 include wherein the variant polypeptide: specifically binds a phosphorylated cyclin E, specifically binds an SCF complex; enhances the ubiquitination of phosphorylated cyclin E; or enhances cellular turnover of cyclin E. The instant polypeptide is referred to herein as an “α-hCdc4 polypeptide variant”.

One embodiment of the present invention provides a polypeptide, comprising (optionally, consisting essentially of) an amino acid sequence that is 95% or more identical to a β-hCdc4 polypeptide set forth in SEQ ID NO:6 and includes an activity substantially similar to SEQ ID NO:6. Examples of the activity substantially similar to SEQ ID NO:6 include wherein the variant polypeptide: specifically binds a phosphorylated cyclin E, specifically binds an SCF complex; enhances the ubiquitination of phosphorylated cyclin E; or enhances cellular turnover of cyclin E. The instant polypeptide is referred to herein as a “β-hCdc4 polypeptide variant”.

In regard to the level or amount of activity, the phrase “activity substantially similar to” in the present embodiments refers to an activity within approximately 50%, or more preferably within approximately 75%, or still more preferably within approximately 90% of a level observed for the wild-type polypeptide.

Candidate α-hCdc4 or β-hCdc4 polypeptide variants can be screened to identify one or more actual variants by determining binding or activity of one of the above biological functions (i.e., a biologically functional equivalent). Such binding and activity assays are disclosed herein, including in the Examples section. Of course, it is preferred to screen those polypeptides meeting the percent identify threshold only.

In general, the greater the percent identity, the higher the probability that a candidate polypeptide will be a biologically functional equivalent of α-hCdc4 or β-hCdc4 as set forth in SEQ ID NO:2 or SEQ ID NO:6, respectively. This arises from the structure-function relationship of proteins. For example, increasing sequence identity generally increases 2 and 3 dimensional structural similarities including binding/active site structural similarities between polypeptides. Accordingly, in certain embodiments, the α-hCdc4 polypeptide variant is 98% or more identical to SEQ ID NO:2 and, more preferably, 99% or more identical to SEQ ID NO:2. in certain embodiments, the β-hCdc4 polypeptide variant is 98% or more identical to SEQ ID NO:6 and, more preferably, 99% or more identical to SEQ ID NO:6.

Herein, sequence alignments and percent identity are determined using JELLYFISH version 1.5 software by LabVelocity (LabVelocity, Inc., San Francisco, Calif.) with the default parameters as follows: ktuple size (1), number of top diagonals (5), window size (5), gap penalty (3), scoring method (percent), weight matrix (Gonnet), gap open penalty (10), gap extension penalty (0.2), residue specific gap penalties (yes), hydrophilic gap benefit (yes), gap separation distance (8), percent of identity for delay (30.0), output order (aligned). Any or all of substitutions, deletions, insertions, additions, gaps, and inclusion of modified amino acid residues (e.g., non-naturally occurring amino acids) are meant to be included in the calculation of percent identity between two sequences. For example, substitution of an allo-isoleucine for a isoleucine or other amino acid residue in one sequence, would reduce the percent identity between sequences when aligned.

Procedures for making polypeptides with substitutions, deletions, insertions, additions, modified residues, etc. are routine in the art, and can be applied to the present polypeptides and variants in light of the present disclosure. One method includes expression of a polypeptide from a polynucleotide encoding such changes (e.g., using site-directed mutagenesis), preferably with purification of the expressed polypeptide. A large number of potential variants can be manufactured using phage display technology. The phage may be screened using a matrix with phospho-polypeptides derived from the region of T62 and/or T380 of cyclin E. A preferred method includes de novo synthesis of the variant polypeptide with the desired alteration(s).

Referring to amino acid substitutions, it is preferred in certain embodiments, that the substitution(s) in an α-hCdc4 or a β-hCdc4 polypeptide variant are conservative substitutions (conservatively substituted). In general, substitution of a first amino acid with an amino acid having a side chain with similar characteristics has a reduced impact on the resulting structure and function of the conservatively substituted variant polypeptide. As used herein, the naturally occurring amino acids are grouped by their side chain characteristics for conservative substitution as follows: aliphatic side chains (G, A, V, L, I); aliphatic side chains with secondary amino group (P); aromatic side chains (F, Y, W); sulfur containing side chains (C and M, except wherein methionine is the first amino acid of a polypeptide); aliphatic hydroxyl side chains (S, T); basic side chains (K, R, H); acidic side chains (D, E, N, Q). For example, alanine may be conservatively substituted for a valine in the non-variant polypeptide. In another example, proline does not have a conservative substitution.

Isolated and Purified Polypeptides

An isolated polypeptide, as used herein, is at least partially removed from other proteins, lipids, carbohydrates, or other materials with which it is naturally associated. The isolated polypeptide comprises, for example, at least a majority of the material in a given sample by dry weight. The terms “purified polypeptide” and “substantially pure polypeptide”, as used herein, each refer to a polypeptide of the present invention that is substantially free of contaminating substances. A polypeptide of the present invention is at least isolated. In general a polypeptide of the present invention comprises a purified polypeptide.

When desired a composition including an isolated or purified polypeptide may further include, but is not limited to: buffer, water, salts, pharmaceutically acceptable carriers, fusions, labels, tags, markers, and protein modifications (e.g., phosphorylation or glycosylation). A purified polypeptide may also include one or more of such agents or be packaged with such agents and still be considered “purified”, as used herein (i.e., these agents are not necessarily contaminating substances). Also, such agents may be added to a purified polypeptide of the invention without introducing contamination. However, with regard to cyclin E, herein non-phosphorylated cyclin E is a contaminant of phosphorylated cyclin E and phosphorylated cyclin E is a contaminant of non-phosphorylated cyclin E.

One skilled in the art can isolate and/or purify a polypeptide of the present invention (including fragments thereof) using standard techniques for protein isolation and purification, in light of the present invention. A substantially pure polypeptide will yield a single major band on a non-reducing polyacrylamide gel, subject to some variation, for example from the presence of a mixed population of the polypeptide (e.g., a portion being phosphorylated or otherwise modified and another portion of the polypeptides not including the modification). Such mixed populations of the polypeptide can be further purified to homogeneity (e.g., thereby providing a pure polyeptide), if desired (e.g., based on charge, size, affinity, or other methods known in the art). The purity of the polypeptide can also be determined, for example, by amino acid sequence analysis.

Non-Naturally Occurring Polypeptides

In optional embodiments, a polypeptide of the present invention comprises a non-naturally occurring polypeptide. Examples of non-naturally occurring polypeptides include, but are not limited to: (a) a polypeptide that includes an amino acid not found in nature or in the organism of interest, such as a synthetic amino acid or amino acid mimetic; (b) a polypeptide that includes a chemical moiety attached to the polypeptide that is not normally associated with the polypeptide in nature, such as a radioactive isotope or a fluorescent marker; (c) a polypeptide that comprises an isolated portion of an amino acid sequence wherein the full sequence occurs in nature, such as a truncated sequence; (d) a polypeptide that does not include a portion of a naturally occurring polypeptide, such as an internal deletion; (e) a fusion polypeptide including two or more amino acid sequences (the same or different sequences) joined together, wherein the amino acid sequences are not found joined together in nature; (f) or an isolated or purified polypeptide removed from the milieu of naturally-occurring substances normally associated with the polypeptide in nature.

Examples of useful modified amino acid residues are provided in the World Intellectual Property Organization (WIPO) Handbook on Industrial Property Information and Documentation, Standard ST.25: Standard for the Presentation of Nucleotide and Amino Acid Sequence Listings in Patent Applications (1998); hereinafter referred to as “WIPO Standard ST.25 of 1998”, incorporated herein by reference. Examples of modified amino acid residues are specifically provided in Appendix 2, Table 2 of the above document.

Manufacture of Polypeptides

In light of the present disclosure and the sequence listing, a polypeptide of this invention can be made using a variety of techniques well known in the art for preparing or making polypeptides in general. For example, by purification from natural sources (e.g., tissue samples or biopsies). In another example, a polypeptide of the present invention can be made through production in a host cell including in bacterial (e.g., K12), eukaryotic, yeast, insect, plant, mammalian, Chinese hamster ovary (CHO), murine, and human cells (e.g., using transfer of a recombinant expression system including those described herein). In still another example, a polypeptide of the present invention is made using de novo synthetic methods well known in the art for sequence specific manufacture of polypeptides. De novo synthesis is convenient for incorporating modified and unusual amino acid residues and residue additions, deletions, and/or substitutions. In preferred embodiments, the polypeptide is isolated or purified.

In one embodiment, an antibody that specifically binds an α-exon 1 hCdc4 polypeptide, or epitope thereof, is used to isolate or purify a polypeptide that includes the antibody epitope (e.g., a polypeptide having an α-exon 1 sequence or immunogenic fragment thereof). Various methods are known for capturing the antibody-antigen complex. For example, the antibody can be isolated or purified on a protein A matrix. In another example, the anti-α-exon 1 antibody is fixed to a matrix for isolation or purification of the α-exon 1 polypeptide.

In another embodiment, the α-exon 1 polypeptide is expressed from a polynucleotide wherein the encoding sequence is fused with a purification tag (e.g., myc-tag, FLAG-tag). The use of purification tags is well known in the art. Numerous vector systems are commercially available for cloning an insert of interest into the vector for expression of the polypeptide of interest fused with a purification or detection tag. The present vectors systems are commercially available for expression in a range of host cell (e.g., bacteria, insect, yeast, mammalian, human) and tailored for expression in specific cell types. Vectors systems are also available for convenient removal of the tag fusion sequence, if desired (e.g., using a peptidase).

In additional embodiments, polypeptide specific antibodies and/or purification tags are used for isolation or purification of β-exon 1 hCdc4, γ-exon 1 hCdc4, and cyclin E polypeptides of the present invention or fragments thereof. In one example, a cyclin E polypeptide that includes a phosphorylation of the T62 residue (cyclin E T62-phospho, described herein) is purified (including purification from a cyclin E without the instant phosphorylation) using an antibody that specifically recognizes cyclin E T62-phospho.

Antibodies

Certain embodiments of the present invention provide antibodies with specific binding activities (e.g., α-hCdc4 specific binding or phospho-cyclin E specific binding). General methods for detecting specific binding of an antibody are well known in the art and can be used to detect specific binding of antibodies of the present invention, in light of the present invention. For example, antibodies of the present invention may be detected using ELISA, immunoprecipitation, immunocytochemistry, affinity capture, and the like. In certain embodiments a primary antibody may be directly detected, or it may be desirable to use a secondary (or higher order) antibody. Secondary antibodies and the like are typically used to increase signal and sensitivity and to decrease noise. An antibody, including those of the present invention, may be labeled sufficient to provide detection capabilities. Compositions and methods for labeling antibodies, in general, and their use in detecting specific binding of the resulting labeled antibody are well known in the art and can be applied to the antibodies of the present invention, in light of the present disclosure. Antibody labels include fluorescent, radioactive, biotin/avidin, and other detectable agents. These may be employed with electrophoresis, mass spectrometry, chromatography and other techniques to resolve the desired compositions. The detection of specific antibody binding includes qualitative, quantitative, and semi-quantitative assay.

Anti-α-Exon 1 Antibody

One embodiment provides an antibody that specifically binds to an hCdc4 α-exon 1 polypeptide. A preferred embodiment provides an antibody that specifically binds to an hCdc4 α-exon 1 comprising the amino acid sequence set forth in SEQ ID NO:4. An even more preferred embodiment provides an antibody that specifically binds to an hCdc4 α-exon 1 comprising the amino acid sequence RGNPSSSQVDEEQ (SEQ ID NO:21). Additional embodiments, provides an antibody that binds a polypeptide consisting essentially of one or more of: an hCdc4 α-exon 1 polypeptide, the amino acid sequence set forth in SEQ ID NO:4, or the amino acid sequence set forth in SEQ ID NO:21; but the antibody does not specifically bind to an amino acid sequence consisting essentially of one or more of: SEQ ID NO:14, an hCdc4 β-exon 1, SEQ ID NO:10, an hCdc4 γ-exon 1, SEQ ID NO:16, a β-hCdc4, SEQ ID NO:6, a γ-hCdc4, or SEQ ID NO:18. An antibody of the present invention is an isolated antibody and preferably is a purified antibody.

Anti-β-Exon 1 Antibody

One embodiment provides an antibody that specifically binds to an hCdc4 β-exon 1 polypeptide. A preferred embodiment provides an antibody that specifically binds to an hCdc4 β-exon 1 comprising the amino acid sequence set forth in SEQ ID NO:10. An even more preferred embodiment provides an antibody that specifically binds to an hCdc4 β-exon 1 comprising the amino acid sequence CQRLPSSRTHGGTESLKG (SEQ ID NO:23). Additional embodiments, provides an antibody that binds one or more of: an hCdc4 β-exon 1 polypeptide, the amino acid sequence set forth in SEQ ID NO:10, or the amino acid sequence set forth in SEQ ID NO:23; but the antibody does not specifically bind to a polypeptide consisting essentially of one or more of: SEQ ID NO:14, an hCdc4 α-exon 1, SEQ ID NO:4, an hCdc4 γ-exon 1, SEQ ID NO:16, an α-hCdc4, SEQ ID NO:2, a γ-hCdc4, or SEQ ID NO:18.

Anti-γ-Exon 1 Antibody

One embodiment provides an antibody that specifically binds to an hCdc4 γ-exon 1 polypeptide. A preferred embodiment provides an antibody that specifically binds to an hCdc4 γ-exon 1 comprising the amino acid sequence set forth in SEQ ID NO:16. An even more preferred embodiment provides an antibody that specifically binds to an hCdc4 γ-exon 1 comprising the amino acid sequence PVDLKSAKEPLPHQTV (SEQ ID NO:24). Additional embodiments, provides an antibody that binds one or more of: an hCdc4 γ-exon 1 polypeptide, the amino acid sequence set forth in SEQ ID NO:16, or the amino acid sequence set forth in SEQ ID NO:24; but the antibody does not specifically bind to a polypeptide consisting essentially of one or more of: SEQ ID NO:14, an hCdc4 α-exon 1, SEQ ID NO:4, an hCdc4 β-exon 1, SEQ ID NO:10, an α-hCdc4, SEQ ID NO:2, a β-hCdc4, or SEQ ID NO:6.

Cyclin E Antibodies

The inventors disclose herein antibodies that specifically bind phospho-cyclin E, but not non-phospho-cyclin E; antibodies that specifically bind phospho-cyclin E and total cyclin E; and antibodies that specifically bind non-phospho-cyclin E, but not phospho-cyclin E. The term “total cyclin E” as used herein includes phosphorylated cyclin E and non-phosphorylated cyclin E. The present antibodies are useful, for example, in diagnostic and screening assays as the inventors discovered that phospho-cyclin E is a tumor marker and is useful to identify tumorous samples independent of cyclin E levels. For instance, numerous tumors include an accumulation of phospho-cyclin E, but total cyclin E content is not necessarily elevated.

One embodiment provides a purified antibody that selectively and specifically binds a phosphorylated cyclin E polypeptide. In other words, a purified antibody that specifically binds a phosphorylated cyclin E polypeptide, but does not specifically bind a non-phosphorylated cyclin E polypeptide.

One embodiment provides a purified antibody that selectively and specifically binds a non-phosphorylated cyclin E polypeptide. In other words, a purified antibody that specifically binds a non-phosphorylated cyclin E polypeptide, but does not specifically bind a phosphorylated cyclin E polypeptide.

One embodiment provides a purified antibody that specifically binds total cyclin E (i.e., a phosphorylated cyclin E and a non-phosphorylated cyclin E). Preferred phosphorylated cyclin E includes T62 phospho-cyclin E and/or T380 phospho-cyclin E.

One embodiment provides a purified antibody that specifically binds to a phosphorylated cyclin E polypeptide. The present antibody is selective for phosphorylated cyclin E and does not specifically bind non-phosphorylated cyclin E.

In one embodiment, the purified antibody specifically binds a T62-phospho-cyclin E polypeptide (a cyclin E polypeptide including a T62 phospho). Preferably, the T62-phospho-cyclin E polypeptide includes 6 or more amino acid residues (optionally 10 to 30 amino acids, 40 or more amino acids, or no more than 40 amino acids). In one embodiment, a purified antibody specifically binds an amino acid sequence consisting essentially of CSLIPTPDKEDDDRV (SEQ ID NO:36), wherein the threonine residue at position six is phosphorylated, but does not specifically bind CSLIPTPDKEDDDRV (SEQ ID NO:37), wherein the threonine residue at position six is not phosphorylated.

In one embodiment, the purified antibody specifically binds a T380-phospho-cyclin E polypeptide (a cyclin E including a T380-phospho). Preferably, the polypeptide includes 6 or more amino acid residues (optionally 10 to 30 amino acids, 40 or more amino acids, or no more than 40 amino acids). In one embodiment, a purified antibody specifically binds an amino acid sequence consisting essentially of SGLLTPPQSGKK (SEQ ID NO:38), wherein the threonine residue at position five is phosphorylated, but does not specifically bind SGLLTPPQSGKK (SEQ ID NO:39), wherein the threonine residue at position five is not phosphorylated.

Manufacture of Antibody

An antibody specific to a polypeptide of the present invention can be made using a variety of techniques well known in the art for preparing or making antibodies in general, in light of the present disclosure and the sequence listing. For example, any polypeptide of the present invention is manufactured, preferably by de novo synthesis, and used to an raise antibodies. One or more antibodies that specifically bind the polypeptide of interest, but do not specifically or significantly bind to other polypeptides are identified using screening methods well known in the art. Antibodies of the present invention may be prepared using a commercial antibody manufacturing service, in light of the present disclosure. An antibody of the present invention can be isolated or purified using an antigen-matrix affinity technique or on a protein A column, for example.

In certain embodiments, it is desirable that the antibody further include a detectable tag (e.g., fluorescent, enzymatic, myc-tag, FLAG-tag, radiolabel, or other such molecule). In certain embodiments, it is desirable that the antibody further include a purification tag (e.g., myc-tag, FLAG-tag, or one of a variety purification tags including those commercially available). Incorporation of detection and purification tags with an antibody are well known in the art.

In certain embodiments, any class or type of antibody is useful including IgG, IgM, monoclonal, polyclonal, Fab, Fab′, F(ab′)2, F(v), single chain Fab, humanized, and the like. It is preferred that the antibody is isolated, substantially purified, purified to homogeneity, recombinant, or otherwise non-naturally occurring. Antibodies are commonly manufactured, for example, in animals (e.g., rabbit, mouse, hamster, sheep, goat, horse, bovine), cell culture (e.g., hybridomas for monoclonal antibody production), bacteria (e.g., phage display), and plants (see e.g., U.S. Pat. No. 6,417,429 to Hein et al., incorporated herein by reference).

In one example, the selective and specific antibodies for phospho- and non-phospho-cyclin E are made by identifying and supplying a commercial antibody production service with appropriate polypeptides sequences and modification information to manufacture the desired antibodies. For instance, the inventors selected: 1) CSLIP(T)PDKEDDDRV (SEQ ID NO:36), wherein the “(T)” refers to a phospho-threonine to manufacture a monoclonal antibody to T62-phospho-cyclin E; 2) CSLIPTPDKEDDDRV (SEQ ID NO:37), wherein the threonine is not phosphorylated to manufacture a monoclonal antibody to cyclin E, wherein T62 is not phosphorylated; 3) C*SGLL(T)PPQSGKK (SEQ ID NO:40), wherein the “(T)” refers to a phospho-threonine to manufacture a monoclonal antibody to T380-phospho-cyclin E (the “C*” refers to a cysteine residue added to the N-terminal of the cyclin E phospho-polypeptide SGLL(T)PPQSGKK (SEQ ID NO:38) for linking to keyhole limpet hemocyanin (KLH); and 4 C*SGLLTPPQSGKK (SEQ ID NO:41), wherein the threonine is not phosphorylated, to manufacture, a monoclonal antibody to cyclin E, wherein T380 is not phosphorylated. The candidate antibodies produced in manufacture can be screen against the present polypeptides to identify specific antibodies with sufficient affinity. The phospho-polypeptides can be manufactured de novo, for example, using solid matrix polypeptide synthesis and phosphorylated, if desired using a variety of kinases and ATP.

Polynucleotides

α-hCdc4 Polynucleotides

One embodiment provides a purified polynucleotide comprising a nucleic acid sequence encoding a polypeptide including an hCdc4 α-exon 1 polypeptide; a α-hCdc4 polypeptide; SEQ ID NO:2; SEQ ID NO:4; SEQ ID NO:4, but not SEQ ID NO:14, or a complement of the nucleic acid sequence. Another embodiment provides a polynucleotide comprising a vector sequence operatively linked to the instant nucleic acid sequence. Still another embodiment, provides a host cell comprising a polynucleotide comprising a vector sequence operatively linked to the instant nucleic acid sequence. A preferred vector comprises an adenoviral vector. It is preferred that the host cell is capable of expressing the polypeptide.

An alternative embodiment provides a purified polynucleotide consisting essentially of a nucleic acid sequence encoding an hCdc4 α-exon 1 polypeptide; a α-hCdc4 polypeptide; SEQ ID NO:2; SEQ ID NO:4; SEQ ID NO:4, but not SEQ ID NO:14, or a complement of the nucleic acid sequence. Another alternative embodiment provides a polynucleotide comprising a vector sequence operatively linked to the instant nucleic acid sequence. A preferred vector comprises an adenoviral vector. Still another alternative embodiment, provides a host cell comprising a polynucleotide comprising a vector sequence operatively linked to the instant nucleic acid sequence. It is preferred that the host cell is capable of expressing the polypeptide.

Certain embodiments provide a purified polynucleotide comprising (optionally, consisting essentially of) a nucleic acid sequence set forth by SEQ ID NO:1; SEQ ID NO:3; or SEQ ID NO:3, but not including SEQ ID NO:13).

A polynucleotide of the present invention includes, for example, deoxyribonucleic acid (DNA) and ribonucleic acid (RNA). One embodiment provides a polynucleotide comprising (or consisting essentially of) a complement of SEQ ID NO:1 or SEQ ID NO:3 wherein the complement is RNA.

Of course, it is understood that the genetic code is degenerate, wherein multiple codons may encode the same amino acid. Equivalent codons (degenerate codons) can be used to make equivalent polynucleotides, each encoding an identical polypeptide. A description and listing of the “The Standard Genetic Code”, including the human genetic code, is provided in Biochemistry, 3rd Edition, Stryer editor, Freeman Publisher (1988) pages 91-115 and inside back cover, incorporated herein by reference. Accordingly, certain embodiments provide degenerate polynucleotide sequences encoding a polypeptide of the present invention.

β-hCdc4 Polynucleotides

One embodiment provides a purified polynucleotide comprising a nucleic acid sequence encoding a polypeptide including an hCdc4 β-exon 1 polypeptide; a β-hCdc4 polypeptide; SEQ ID NO:6; SEQ ID NO:10; SEQ ID NO:10, but not SEQ ID NO:14, or a complement of the nucleic acid sequence. Another embodiment provides a polynucleotide comprising a vector sequence operatively linked to the instant nucleic acid sequence. A preferred vector comprises an adenoviral vector. Still another embodiment, provides a host cell comprising a polynucleotide comprising a vector sequence operatively linked to the instant nucleic acid sequence. It is preferred that the host cell is capable of expressing the polypeptide.

An alternative embodiment provides a purified polynucleotide consisting essentially of a nucleic acid sequence encoding an hCdc4 β-exon 1 polypeptide; a β-hCdc4 polypeptide; SEQ ID NO:6; SEQ ID NO:10; SEQ ID NO:10, but not SEQ ID NO:14, or a complement of the nucleic acid sequence. Another alternative embodiment provides a polynucleotide comprising a vector sequence operatively linked to the instant nucleic acid sequence. A preferred vector comprises an adenoviral vector. Still another alternative embodiment, provides a host cell comprising a polynucleotide comprising a vector sequence operatively linked to the instant nucleic acid sequence. It is preferred that the host cell is capable of expressing the polypeptide.

Certain embodiments provide a purified polynucleotide comprising (optionally, consisting essentially of) a nucleic acid sequence set forth by SEQ ID NO:5; SEQ ID NO:9; or SEQ ID NO:9, but not including SEQ ID NO:13). One embodiment provides a polynucleotide comprising (or consisting essentially of) a complement of SEQ ID NO:5 or SEQ ID NO:9 wherein the complement is RNA.

Variants

One embodiment of the present invention provides a polynucleotide, comprising (optionally, consisting essentially of) a nucleic acid sequence encoding an amino acid sequence that is 95% or more identical to an α-hCdc4 polypeptide set forth in SEQ ID NO:2 and includes a biological activity of α-hCdc4 as disclosed herein. Preferably, the amino acid sequence is 98% or more (and even more preferably 99% or more) identical to an α-hCdc4 polypeptide.

One embodiment of the present invention provides a polynucleotide, comprising (optionally, consisting essentially of) a nucleic acid sequence encoding an amino acid sequence that is 95% or more identical to a β-hCdc4 polypeptide set forth in SEQ ID NO:6 and includes a biological activity of β-hCdc4 as disclosed herein. Preferably, the amino acid sequence is 98% or more (and even more preferably 99% or more) identical to a β-hCdc4 polypeptide.

Vectors

In certain preferred embodiments, a polynucleotide further includes a vector sequence operatively linked to a nucleic acid sequence set forth in an embodiment of the present invention of the present invention. As used herein, the term “insert” or “nucleic acid insert” is meant to refer to a nucleic acid sequence or polynucleotide embodied by the present invention (including the sequence listing), not to nucleic acid sequences in general.

A polynucleotide including a vector with an operably linked insert is useful, for example, in producing copies of the nucleic acid sequence, in manufacturing an expression product (e.g., polypeptides of the present invention), in administering a polypeptide of the present invention to a cell (e.g., by transfection and expression of the encoding nucleic acid sequence), and in providing convenient cloning tools (e.g., a “shuttle vector”). As used herein, the terms “expression vector” and “expression construct” can be used interchangeably.

Expression includes, but is not limited to: transcription of an RNA from a DNA, translation of a polypeptide from an RNA, or both. Typically, a control element is operably linked to at least one polynucleotide sequence and modulates the generation of one or more expression product(s) from the insert. In general, the control element is included in the vector sequence, but certain embodiments provide that one or more control element is included in the insert. Expression may be produced in a cell (e.g., in vivo), in vitro, or using any other manner known in the art (e.g., ex vivo). Commercially available kits are particularly useful for in vitro expression.

Control elements include, but are not limited to one or more of a(n): promoter, enhancer, TATA box, start (initiator) codon, Shine-Dalgarno sequence, polyadenylation site (poly A site), silencer, terminator, stop codon, signal sequence, internal ribosome entry site (IRES), intron, splice donor, splice acceptor, and branch point. Useful vector sequences for making and using compositions of the present invention are commercially available from numerous manufactures. Typically, one will select a vector based upon the published characteristics and utilities of each vector. Table 1 provides certain examples of useful vectors.

TABLE 1
Examples of Useful Vectors

| | Preferred Host | | |
|---|---|---|---|
| Vector | Cell(s) | Tag/Excisable | Company |
| pUC8 | E. coli | | BioRad Laboratories |
| | | | (Richmond, CA) |
| pUC9 | E. coli | | BioRad Laboratories |
| pBR322 | E. coli | | BioRad Laboratories |
| pBR329 | E. coli | | BioRad Laboratories |
| pPL | E. coli | | Pharmacia |
| pKK233 | JM109 | | Pharmacia |
| | | | (Piscataway, NJ) |
| pQE-Express | M15 or SG13009 | 6XHis | Qiagen |
| | | | (Valencia, CA) |
| pCYB1-4 | E. coli | Chitin Binding Domain | New England Biolab |
| | | Excisable/Intein | (Beverly, MA) |
| pBAD/myc-His | LMG194, Top10 | 6XHis, myc-tag | Invitrogen |
| | | | (Carlsbad, CA) |
| pBAD/myc-His ABC | Prokaryotic | 6XHis, Anti-Xpress-Epitope | Invitrogen |
| | | Excision/Enterokinase | |
| pTet-Off | Mammalian (e.g., | | BD Biosciences-Clontech |
| pTet-On | 293, MCF7, | | (Palo Alto, CA) |
| | HeLa, CHO | | |
| Adeno-X | Mammalian | | Clontech |
| Expression System | | | |
| Retro-X System | Mammalian | | Clontech |

Certain promoters, enhancers, and promoter/inducing agent combinations useful for expression of a nucleic acid insert of the present invention are set forth in U.S. Pat. No. 6,331,284 to Hung et al., incorporated herein by reference. Numerous inducible expression systems are commercially available. For example, the COMPLETE CONTROL Inducible Mammalian Expression System and LACSWITCH II Inducible Mammalian Expression System (Stratagene), the ZAP EXPRESS vector (mammalian expression and inducible prokaryotic expression using IPTG, Stratagene), and the ADENOVIRUS-X TET-OFF expression system (includes controllable expression in mammalian cells from an adenovirus derived vector Clontech). Alternatively, any promoter/enhancer combination (as per the Eukaryotic Promoter Data Base EPDB) could also be used to drive expression of a nucleic acid sequence of the present invention.

Modulating expression includes: increasing, decreasing, and maintaining production of an expression product. For example, production of a gene product can be increased in response to a stimulus (e.g., inducible expression, including tissue specific expression), decreased, and/or maintained. Expression includes, but is not limited to: basal expression, constitutive expression, inducible expression, tissue specific expression, and cell cycle specific expression.

TABLE 2
Examples of Useful Control Elements, Vector Sequences, and/or
Expression Systems for Certain Desirable Expression Patterns

| Desired Expression | |
|---|---|
| Characteristic | Examples of Useful Elements, Vectors, Systems |
| Basal | Cytomegalovirus (CMV) promoter; CMV based vectors |
| Constitutive | RSV LTR in EBV vectors from CLONTECH |
| Inducible | Ecdysone-inducible mammalian expression system from |
| | INVITROGEN; pBl-L-tet (luciferase gene allows for |
| | transfection/expression assay when polypeptide of interest does |
| | not include activity), CLONTECH |
| Tissue specific | Macrophage scavenger receptor upstream elements; pBC1 Milk |
| | Expression Vector, INVITROGEN |
| Cell cycle specific | Certain cyclin promoters and enhancers (e.g., cyclin A and |
| | cyclin E) |

The particular promoter that is employed to control the expression of a polynucleotide herein is not believed to be critical, so long as it is capable of expressing the polynucleotide in the targeted cell at sufficient levels for the desired embodiment. For example, where expression in a human cell is desired, it is preferable to position the polynucleotide coding region adjacent to and under the control of a promoter that is capable of being expressed in a human cell. Generally speaking, such a promoter might include either a human or a viral promoter.

In certain embodiments, the human CMV immediate early gene promoter, the simian virus 40 (SV40) early promoter, or the Rous sarcoma virus long terminal repeat (RSV-LTR) can be used to obtain high-level expression of the polynucleotide.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of polynucleotides of the present invention.

Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase (CAT) (prokaryotic) may be employed. Immunologic markers also can be employed. The selectable marker employed is not believed to be important, so long as it is capable of being expressed along with the nucleic acid insert of the present invention. Further examples of selectable markers are well known to one of skill in the art.

In certain embodiment, the expression construct comprises a virus or engineered construct derived from a viral genome. The ability of certain viruses to enter cells via receptor-mediated endocytosis and, in some cases, integrate into the host cell chromosomes, have made them attractive candidates for polynucleotide or gene transfer into mammalian cells. Useful viral vectors include modified adenovirus and retrovirus vectors known in the art for transfer and expression of polynucleotides in host cells. A viral vector comprises an adenoviral or adenoviral derived vector (see, e.g., Strohmaier et al (2001) Nature 413:316-322 and Mercurio et al (1994) Proc Natl. Acad. Sci. USA 91:8802-8806). In addition, uptake of naked DNA, especially in muscle cells, and receptor-mediated (or histone mediated) uptake of DNA/ligand complexes is contemplated. Accordingly, expression vectors need not be viral but, instead, may be any plasmid, cosmid or phage construct that is capable of supporting expression of encoded genes in mammalian cells, such as pUC or BLUESCRIPT plasmid series.

Host Cells

One embodiment provides an isolated host cell comprising a polynucleotide of the present invention. A preferred embodiment provides a host cell comprising a polynucleotide, the polynucleotide including a vector sequence operably linked to a nucleic acid insert encoding a polypeptide of the present invention and capable of expressing the polypeptide. Certain useful host cells include, but are not limited to: bacteria (e.g., K12), eukaryotic, yeast, insect, plant, mammalian, Chinese hamster ovary (CHO), 293, KB, murine, and human cells. It is preferred that the vector sequence includes sufficient control elements for expression of a polypeptide encoded by the nucleic acid insert in the desired cell(s). Certain control elements may also be included to inhibit expression as desirable (e.g., TET-OFF, Clontech). In certain embodiments, a host cell is useful for producing a polypeptide for isolation or administration. The term “host cell” includes any progeny of the subject host cell.

In certain preferred embodiments, a host cell is capable of expressing an α-exon 1 (e.g., SEQ ID NO:4), β-exon 1 (e.g., SEQ ID NO:10), a γ-exon 1 (e.g., SEQ ID NO:16), an α-hCdc4 (e.g., SEQ ID NO:2), a β-hCdc4 (e.g., SEQ ID NO:6), a γ-hCdc4 (e.g., SEQ ID NO:18), a cyclin E including a mutation inhibiting phosphorylation (e.g., SEQ ID NO:46, SEQ ID NO:47, or SEQ ID NO:48).

One embodiment provides a host cell having a defect in expression of a native hCdc4 polypeptide, the host cell comprising a non-native (or non-naturally occurring) polynucleotide encoding an hCdc4 polypeptide and capable of expressing the hCdc4 polypeptide. It is preferred that the polynucleotide is included in a vector including sufficient control elements for expression of the encoded hCdc4 polypeptide. A preferred host cell comprises SUM149PT. SUM149PT includes a genetic defect that essentially eliminates expression of wild-type hCdc4 (including α-, β-, and γ-isoforms) and essentially eliminates native wild-type hCdc4 activity (see Loss of hCdc4 in the Examples section).

One use of an hCdc4 defective host cell is in a host cell complementation assay. A “host cell complementation assay”, as used herein, comprises introducing a polynucleotide encoding a putative hCdc4 polypeptide, wherein the polynucleotide includes sufficient elements to effect expression of the putative hCdc4 polypeptide in the host cell. Such assay is useful, for example, to determine if the encoded polypeptide comprises an hCdc4 variant (e.g., having a significant activity of an hCdc4 polypeptide and 95% or more sequence identity) or a mutant (e.g., lacking a significant activity of an hCdc4 polypeptide, but having 95% or more sequence identity).

Accordingly, in one embodiment, a host cell, having a defect in native hCdc4 activity, includes an exogenous polynucleotide including a nucleic acid sequence encoding an hCdc4 polypeptide, wherein host cell is capable of expressing non-native hCdc4 from the exogenously introduced polynucleotide (alternatively, the exogenous polynucleotide is capable of expressing a non-native hCdc4 in the host cell). The exogenous polypeptide may, for example, be incorporated into the genome of the host cell using techniques known to one of ordinary skill in the art. In certain alternative embodiments, the host cell comprises a cell having an elevated cyclin E level. More preferably, the host cell includes an accumulation in phosphorylated cyclin E (or, optionally, a substantial increase in phosphorylated cyclin E to non-phosphorylated cyclin E relative to a non-transformed cell, preferably 184A1 cells (an immortalized, non-transformed breast epithelial cell line). In one embodiment, the host cell comprises a human tumor cell having a defect in an hCdc4 activity. Preferably, the tumor cell further includes an elevated level of cyclin E and, more preferably, an accumulation of phospho-cyclin E. In certain embodiments, it may be desirable to use an inducible or controlled expression system (e.g., see Vector section).

In one embodiment, expression of an exogenous hCdc4 in a host cell having a defect in native hCdc4 expression is useful in a method of testing an activity of the exogenous (administered) hCdc4. For example, one may desire to make a variant or mutant hCdc4. A recombinant hCdc4 polynucleotide capable of expressing hCdc4 in the host cell can be made, administered to the host cell, and the activity of the expressed hCdc4 can be determined. Measurements of hCdc4 activity, for example, can reveal that a particular hCdc4 polypeptide (or underlying polynucleotide) is an hCdc4 variant in that it includes a substantially wild-type activity (and includes other features of a variant). Alternatively, the exogenous hCdc4 can be fused or otherwise operably linked with a membrane transport sequence and the hCdc4 polypeptide can be directly administered to the cell. In another alternative, the exogenous hCdc4 polypeptide can be combined with lipids or liposomes known in the art to be effective transfer reagents and administer to the cell in this manner.

Mutants

Certain embodiments of the present invention provide, mutants of α-hCdc4, β-hCdc4, and cyclin E polynucleotides and polypeptide that are expressed therefrom. A mutant α-hCdc4 or β-hCdc4 can be identified, for example, by assaying one or more of 1) specific binding to phosphorylated wild-type cyclin E; 2) specific binding with the SCF complex; 3) activity, preferably specific activity, in a cyclin E ubiquitination or proteolysis assay; and 4) presence in a tumor. A mutant α-hCdc4 or β-hCdc4, as opposed to a variant, will show a substantially reduced binding or activity in at least one such assay (e.g., more than ½ reduction, preferably more than a ¾ reduction, and most preferably essentially eliminated). The identification of a somatic mutant of α-hCdc4 or β-hCdc4 in a tumor can determined by comparison to normal fluid or tissue of the subject. The identification of a genetic mutation can be identified, for example, by observing genomic rearrangement; defective splicing; and abnormal expression of mRNA and/or protein, including reduced levels of expression or abnormal lengths or pattern of expression in various tissues. A mutant cyclin E can be identified, for example, by a reduction, preferably an elimination, of phosphorylation of a T62 and/or a T380 site as disclosed herein.

One embodiment provides a purified polynucleotide comprising a mutant α-hCdc4 polynucleotide including a mutation in a codon selected from the group consisting essentially of: 124, 367, 371, 465, 472, 479, or 658 of the polynucleotide, wherein a wild-type α-hCdc4 polynucleotide comprises a nucleic acid sequence set forth in SEQ ID NO:1 and the codon numbering is relative to SEQ ID NO:1 (accounting for the fact that codons include 3 nucleic acid residues). The inventors have identified each of these mutant α-hCdc4 polynucleotides in endometrial cancer cells (see the Examples section). One example of a use of a mutant α-hCdc4 polynucleotide is for the manufacture of mutant α-hCdc4 polypeptides associated with tumors. The mutant α-hCdc4 polypeptide, or immunogenic fragment thereof, is useful as a control in an assay to detect tumorigenesis or tumor cells, for example. Accordingly, one embodiment provides a purified polypeptide (or, optionally, an immunogenic fragment thereof) expressed from the mutant α-hCdc4 polynucleotide, wherein a wild-type substantially full length α-hCdc4 polypeptide comprises an amino acid sequence set forth in SEQ ID NO:2.

One embodiment provides a purified polypeptide comprising a mutant α-hCdc4 polypeptide including a truncation mutation or a frame shift mutation in a C-terminal domain of the mutant α-hCdc4 polypeptide, wherein a wild-type α-hCdc4 polypeptide includes an amino acid sequence set forth in SEQ ID NO:2, and wherein a wild-type C terminal domain includes a sequence set forth in SEQ ID NO:14. In one embodiment, the truncation mutation or the frame shift mutation occurs at amino acid position 168 or greater relative to the wild-type α-hCdc4 polypeptide. In one embodiment, the truncation mutation or the frame shift mutation occurs at amino acid position 284 or greater relative to the wild-type α-hCdc4 polypeptide (optionally, in the region of the F-box domain, for example, substantially within SEQ ID NO:19). In one embodiment, the truncation mutation or the frame shift mutation occurs at amino acid position 369 or greater relative to the wild-type α-hCdc4 polypeptide (optionally, in the region including the WD40 repeats, for example, substantially within SEQ ID NO:20).

One embodiment provides a purified polynucleotide comprising a mutant β-hCdc4 polynucleotide including a mutation at codon 23 of the polynucleotide, wherein a wild-type β-hCdc4 polynucleotide comprises a nucleic acid sequence set forth in SEQ ID NO:5. This mutation was identified by the inventors in cells derived from an endometrial tumor (see Examples section). Another embodiment provides a polypeptide manufactured by expression of the instant polynucleotide, wherein a wild-type α-hCdc4 polypeptide comprises an amino acid sequence set forth in SEQ ID NO:6. It is preferred that polypeptides of the present invention are purified.

One embodiment provides a purified polypeptide comprising a mutant β-hCdc4 polypeptide including a truncation mutation or a frame shift mutation in a C-terminal domain of the mutant β-hCdc4 polypeptide, wherein a wild-type β-hCdc4 polypeptide includes an amino acid sequence set forth in SEQ ID NO:6, and wherein a wild-type C terminal domain includes a sequence set forth in SEQ ID NO:14. In one embodiment, the truncation mutation or the frame shift mutation occurs at amino acid position 88 or greater relative to the wild-type β-hCdc4 polypeptide. In one embodiment, the truncation mutation or the frame shift mutation occurs at amino acid position 204 or greater relative to the wild-type α-hCdc4 polypeptide (or, optionally, within the F-box region). In one embodiment, the truncation mutation or the frame shift mutation occurs at amino acid position 289 or greater relative to the wild-type α-hCdc4 polypeptide (or, optionally, in the WD40 region).

One embodiment provides a purified polynucleotide comprising a nucleic acid sequence encoding a cyclin E polypeptide including a T62 mutation, wherein a wild-type cyclin E polypeptide includes an amino acid sequence set forth in SEQ ID NO:31 or SEQ ID NO:32 (e.g., the mutation of the polypeptide is at amino acid position 62 of SEQ ID NO:32 or amino acid position 77 of SEQ ID NO:31). It is preferred that the mutation is a substitution mutation or a deletion mutation. Another embodiment provides polypeptide manufactured by expression of the instant polynucleotide, preferably the polypeptide is purified. A preferred mutation is a threonine to alanine substitution (T62A).

One embodiment provides a polynucleotide comprising a nucleic acid sequence encoding a cyclin E polypeptide including a T62 mutation and further including a T380 mutation. A T380 mutation of the polypeptide, for example, is at amino acid position 380 of SEQ ID NO:32 or amino acid position 395 of SEQ ID NO:31. Another embodiment provides a polypeptide manufactured by expression of the instant polynucleotide. A preferred mutation is a threonine to adenine substitution at T62 and T380 (T62A and T380A). Another embodiment provides polypeptide manufactured by expression of the instant polynucleotide, preferably the polypeptide is purified.

Biologically Active Agent and Fusions

One embodiment provides a polypeptide (alternatively, a composition including a polypeptide) of the present invention further including a biologically active agent.

As used herein, a preferred operable linkage for combining amino acid sequences of the present invention comprises a peptide bond. Alternatively, the amino acid sequences are combined by any method known to one of skill in the art including, but not limited to: a covalent bond, a non-covalent bond, an ionic bond, charge to charge association, linkage of each sequence to members of a binding complex wherein the members associate and bring the sequences into proximity, an amino acid polymer (a linking amino acid sequence, wherein the sequences are preferably linked by peptide bonds), and chemical crosslinking.

In certain embodiments, it is desirable to combine a polypeptide with a detectable marker. Examples of useful detectable markers include, but are not limited to: antibody recognized moieties (e.g., FLAG epitope (DYKDDDDK SEQ ID NO:); c-myc epitope (EQKLISEEDL); and digoxigenin), visualization markers (e.g., green fluorescent protein, luciferase, radiolabels, and biotin/avidin), enzymatic markers (e.g., horseradish peroxidase), and combinations thereof (e.g., radiolabeled or enzyme labeled antibodies). Examples of useful radiolabels include, but are not limited to: 125I, 14C, 3H, 35S, 33P, and 32P. Many additional detectable markers and methods of use with polypeptides in general, are known in the art. Such detection agents can be combined and used with a polypeptide of the present invention, in light of the present invention. In certain embodiments, it is desirable to operably linked a polypeptide set forth in an embodiment of the present invention with another polypeptide to form a fusion polypeptide including the polypeptide of the present invention.

Methods of Detecting

α-Isoform

One embodiment provides a method of identifying an α-hCdc4 expression product in a sample, comprising specifically detecting an α-exon 1 sequence in the sample, wherein the α-exon 1 sequence is a ribonucleic acid sequence or an amino acid sequence. In a preferred embodiment, the α-exon 1 ribonucleic acid sequence is set forth in SEQ ID NO:28. In an optional embodiment, the α-exon 1 ribonucleic acid sequence is a fragment of the sequence set forth in SEQ ID NO:28 having 10 or more consecutive ribonucleic acid residues of SEQ ID NO:28. In another preferred embodiment, the α-exon 1 amino acid sequence is set forth in SEQ ID NO:4. In an optional embodiment, the α-exon 1 amino acid sequence is a fragment of the sequence set forth in SEQ ID NO:4 having 6 or more consecutive amino acid residues of SEQ ID NO:4.

One embodiment provides a probe for specifically detecting an α-exon 1 sequence. In one embodiment, the probe comprises a polynucleotide that is complementary to 10 or more consecutive residues of SEQ ID NO:28 and specifically binds SEQ ID NO:28 under stringent hybridization conditions. In another embodiment, the probe comprises an antibody that specifically binds SEQ ID NO:4. It is preferred that the probe is operably linked with a detectable agent (e.g., a radionucleotide, a fluorescent molecule, an enzyme, a capture molecule, or the like).

β-Isoform

One embodiment provides a method of identifying a β-hCdc4 expression product in a sample, comprising specifically detecting a β-exon 1 sequence in the sample, wherein the β-exon 1 sequence is a ribonucleic acid sequence or an amino acid sequence. In a preferred embodiment, the β-exon 1 ribonucleic acid sequence is set forth in SEQ ID NO:29. In an optional embodiment, the β-exon 1 ribonucleic acid sequence is a fragment of the sequence set forth in SEQ ID NO:29 having 10 or more consecutive ribonucleic acid residues of SEQ ID NO:29. In another preferred embodiment, the β-exon 1 amino acid sequence is set forth in SEQ ID NO:10. In an optional embodiment, the β-exon 1 amino acid sequence is a fragment of the sequence set forth in SEQ ID NO:10 having 6 or more consecutive amino acid residues of SEQ ID NO:10.

One embodiment provides a probe for specifically detecting a β-exon 1 sequence. In one embodiment, the probe comprises a polynucleotide that is complementary to 10 or more consecutive residues of SEQ ID NO:29 and specifically binds SEQ ID NO:29 under stringent hybridization conditions. In another embodiment, the probe comprises an antibody that specifically binds SEQ ID NO:10. It is preferred that the probe is operably linked with a detectable agent.

γ-Isoform

One embodiment provides a method of identifying a γ-hCdc4 expression product in a sample, comprising specifically detecting a γ-exon 1 sequence in the sample, wherein the γ-exon 1 sequence is a ribonucleic acid sequence or an amino acid sequence. In a preferred embodiment, the γ-exon 1 ribonucleic acid sequence is set forth in SEQ ID NO:30. In an optional embodiment, the γ-exon 1 ribonucleic acid sequence is a fragment of the sequence set forth in SEQ ID NO:30 having 10 or more consecutive ribonucleic acid residues of SEQ ID NO:30. In another preferred embodiment, the γ-exon 1 amino acid sequence is set forth in SEQ ID NO:16. In an optional embodiment, the γ-exon 1 amino acid sequence is a fragment of the sequence set forth in SEQ ID NO:16 having 6 or more consecutive amino acid residues of SEQ ID NO:16.

One embodiment provides a probe for specifically detecting a γ-exon 1 sequence. In one embodiment, the probe comprises a polynucleotide that is complementary to 10 or more consecutive residues of SEQ ID NO:30 and specifically binds SEQ ID NO:30 under stringent hybridization conditions. In another embodiment, the probe comprises an antibody that specifically binds SEQ ID NO:16. It is preferred that the probe is operably linked with a detectable agent.

Phosphorylated Cyclin E

One embodiment provides a method of detecting a phosphorylated cyclin E in a sample comprising contacting the sample with an anti-phospho cyclin E antibody and detecting a specific binding of the antibody, thereby detecting the phospho-cyclin E in the sample. The anti-phospho cyclin E antibody is specific to phosphorylated cyclin E, wherein the cyclin E is phosphorylated at T62, T380, or both. In one embodiment, the antibody specifically binds phospho-T62 cyclin E (cyclin E phosphorylated at threonine 62). In another embodiment, the antibody specifically binds phospho-T380 cyclin E. In still another embodiment, the antibody specifically binds a T62-phospho and a T380-phospho site of cyclin E (e.g., a multivalent antibody, or binding fragments thereof).

One embodiment provides a method of detecting a phospho-cyclin E in a sample, comprising contacting the sample with an antibody that specifically binds a polypeptide comprising (alternatively, consisting essentially of) an amino acid sequence CSLIPTPDKEDDDRV (SEQ ID NO:36), wherein the threonine residue is phosphorylated; and detecting a specific binding of the antibody, thereby detecting the phospho-cyclin E in the sample. This embodiment further provides a method of detecting a T62-phospho-cyclin E.

One embodiment provides a method of detecting a phospho-cyclin E in a sample, comprising contacting the sample with an antibody that specifically binds a polypeptide comprising (alternatively, consisting essentially of) an amino acid sequence SGLLTPPQSGKK (SEQ ID NO:38), wherein the threonine residue is phosphorylated; and detecting a specific binding of the antibody, thereby detecting the phospho-cyclin E in the sample. This embodiment further provides a method of detecting a T380-phospho-cyclin E.

In general, a sample is any material for which it is desired to detect or measure a phospho-cyclin E content. Samples may include mammalian cells, tissues, or bodily fluid. Preferred samples include human cells, tissues, or bodily fluids. For example, certain sample are derived from the lung, breast, epidermis, intestine, liver, stomach, endometrium, prostate, bladder, cervix, uterus, vagina, testicle, blood, lymph, serum, plasma, mesoderm, endoderm, or ectoderm. In another example, samples are obtained from cells or fluids that are suspected of or known to come from a tumor. In general, samples are obtained using methods well known in the art. Such methods may include biopsy or lavage.

It is understood that the antibody is contacted to the sample under conditions suitable for antibody binding reactions including the use of solutions, buffers (including physiological buffers that mimic conditions in bodily fluids or in the bodily fluids themselves with or without a buffer agent), reaction times, temperatures, pH, stabilizers, blocking agents, and the like suitable for antibody binding. Antibody binding conditions are well known in the art and can be applied to assays of the present invention, in light of the present invention.

One embodiment provides a method to measure the ratio of phosphorylated to non-phosphorylated cyclin E in a sample, comprising contacting the sample with an antibody specific to non-phosphorylated (preferably T62 and/or T380) cyclin E; contacting the sample with another antibody specific to phosphorylated cyclin E (preferably T62-phospho and/or T380-phospho); detecting a specific binding of each antibody; and determining the ratio of specific binding of the first antibody to the specific binding of the second antibody. In certain embodiments, a high ratio of phospho-cyclin E to cyclin E is a marker for deregulated cyclin E turnover; neoplasia, and tumorigenesis, and correlates with tumor grade and metastasis in tumors. Another embodiment, a ratio of phospho-cyclin E to total cyclin E (phosphorylated and non-phosphorylated cyclin E) is determined.

In a preferred embodiment, the present methods are useful to compare the relative phospho-cyclin E content, or ratio, in tissue samples using immunocytochemical techniques. In another embodiment, the present methods are useful to compare the relative phospho-cyclin E content in various samples using Western blotting techniques. In certain embodiments, it is preferred to compare the accumulation of phospho-cyclin E of the ratio of phospho-cyclin E/non-phospho-cyclin E to normal tissues from the same subject or to a previously selected standard value.

In general, non-tumor cells do not contain detectable phospho-cyclin E. Thus, the current invention provides the ability to detect or confirm a tumorigenic transformation. The present invention discloses that the ratio of phospho-cyclin E to total cyclin E or non-phospho-cyclin E is positively correlated with tumor aggressiveness (e.g., stage or grade) and metastasis. Thus, the current invention provides a prognostic indicator for tumor outcome for a patient with a tumor. Having a prognosis is important to designing a therapeutic regimen for a patient with a tumor. For example, patients with a poor tumor prognosis typically receive aggressive therapy. While patients with a good prognosis typically a less intensive therapy. In another example, patients with an increased phospho-cyclin E or phospho-cyclin E ratio in the tumor tissue would receive more intensive therapy than patients with no substantive phospho-cyclin E or phospho-cyclin E ratio.

Tumor

The inventors made certain discoveries including: 1) defects in the hCDC4 gene, or expression thereof, in 16% or more of endometrial tumors tested; 2) the hCdc4 gene is located at human chromosome locus 4q32; 3) a correlation between loss of heterogeneity (LOH) at the 4q32 locus and defects in the hCdc4 gene or expression; 4) a correlation between defects in the hCdc4 gene or expression and accumulation of cellular phospho-cyclin E; 5) a correlation between an accumulation of phospho-cyclin E and endometrial tumors; 6) a positive correlation between the phospho-cyclin E over total or non-phospho-cyclin E and prognosis of a tumor (e.g., tumor grade, stage, metastasis, and/or potential to metastasize) and 6) a correlation between an accumulation of phospho-cyclin E and LOH at the 4q32 locus.

Deletions of the 4q32 region are reported in 31% of all neoplasms including 67% of lung cancers, 63% of head and neck cancers, 41% testicular cancers, and 27% of breast cancers. Remarkably, the 16% or more frequency of hCDC4 gene alterations identified in endometrial adenocarcinomas disclosed in this application is comparable to the frequency (17%) of 4q32 deletion reported for endometrial adenocarcinomas elsewhere (see Knuutila, S. et al. (1999) Am J Pathol 155:683-694).

Accordingly, it is an embodiment of the present invention that defects in hCdc4 correlate with neoplasia and tumors, in general. For example, defects in hCdc4 are correlated with endometrial adenocarcinoma. In another example, defects in hCdc4 are associated with such tumors including: lung, head and neck, testicular, and breast tumors. Certain preferred samples include human cells, tissues, or bodily fluids. For example, sample may be derived from the lung, breast, epidermis, intestine, liver, stomach, endometrium, prostate, bladder, cervix, uterus, vagina, testicle, blood, lymph, serum, plasma, mesoderm, endoderm, or ectoderm. Samples may be obtained, for example, through lavage, biopsy, aspiration (e.g., with a needle), or generally any method known in the art. In certain embodiments, a subject is a mammal and, preferably, a human suspected of having a tumor.

It is a another embodiment that the accumulation of phospho-cyclin E, or a significant ratio of phospho-cyclin E/non-phospho-cyclin E in a cell, is a tumor marker independent of total cyclin E levels (see Table 9 in the Examples section).

One embodiment provides a method of detecting a tumor in a subject, comprising: contacting a sample of the subject with an antibody that specifically binds to a phosphorylated cyclin E; and detecting a specific binding of the antibody, thereby detecting the tumor cell. It is preferred that the antibody specifically binds to a T62-phospho cyclin E polypeptide. Alternatively, the antibody specifically binds to a T380-phospho cyclin E polypeptide. Optionally, the antibody specifically binds to a T62-phospho cyclin E epitope and a T380-phospho cyclin E epitope. A preferred T62-phospho epitope comprises (or, optionally consists essentially of) CSLIPTPDKEDDDRV (SEQ ID NO:36), wherein the threonine residue of SEQ ID NO:36 is phosphorylated. A preferred T380-phospho epitope comprises (or, optionally consists essentially of SGLLTPPQSGKK (SEQ ID NO:38), wherein the threonine residue of SEQ ID NO:38 is phosphorylated. The present method includes detecting a tumor phenotype in a sample derived from a tumor or suspected of being derived from a tumor. A preferred tumor comprises an endometrial tumor. Alternative a tumor comprises a tumor of the lung, head and neck, testical, or mammary gland.

One embodiment provides a method of detecting a tumor in a subject, comprising: screening for a defect in an hCdc4 gene, mRNA, or polypeptide activity.

For example, loss of an α-hCdc4 mRNA, a β-hCdc4 mRNA, or a γ-hCdc4 mRNA; preferably, an α-hCdc4 mRNA can be screened by SSCP analysis of amplified mRNAs and electrophoresis. A loss of the approximately 5.5 kb α-hCdc4 mRNA or 4 kb β-hCdc4/γ-hCdc4 mRNAs indicates the loss of the α-hCdc4 mRNA, a β-hCdc4 mRNA, or a γ-hCdc4 mRNA; respectively. A shift in band mobility can also indicate a loss of mRNA expression (e.g., to 11 kb as observed in an embodiment set forth in the Examples section). Sequences SEQ ID NO:42, SEQ ID NO:43, SEQ ID NO:44, and SEQ ID NO:45 are useful PCR primers and can be used as labeled probes.

In anther example, a defect in an hCdc4 gene locus can be determined using PCR or cloning techniques and sequencing techniques well known in the art to determine the sequence of the coding exons of an hCdc4 gene from a particular sample. If no exon or sequence is obtained, the region can be examined for LOH at the 4q32 locus which also correlates with defects in the hCdc4 gene.

In still another example, a defect in an hCdc4 polypeptide activity can be detected by isolating hCdc4 polypeptide from a sample (e.g., using affinity capture with hCdc4 specific antibodies, preferably hCdc4 isoform specific antibodies) and analyzing the hCdc4 polypeptide, or isoform, for an hCdc4 activity as disclosed herein (e.g., binding to phosphorylated cyclin E).

One embodiment provides a method of determining a relative prognosis of a patient having a tumor, comprising screening for a defect in hCdc4 (including genetic or expression defect; preferably α-hCdc4), an accumulation in phospho-cyclin E, or an abnormal phospho-cyclin E ratio (ratio of phospho-cyclin E over total cyclin E or non-phospho-cyclin E); and determining a relatively poor prognosis when an hCdc4 defect, an accumulation in phospho-cyclin E, or an abnormal phospho-cyclin E ratio is detected. A tumor can be identified in a patient, for example, by morphological methods well known in the art; such as the formation of a mass of hyperproliferating or necrotic cells (e.g., solid tumors) or an increase in specific populations of blood cells or lymphocytes (alternatively; diffuse tumors, hematological tumors, or soft tissue tumors).

### EXAMPLES

SCF Modulates Cyclin E Turnover in Yeast

In yeast, a protein-ubiquitin ligase system known as SCF targets a number of proteins for ubiquitin-mediated proteolysis in a phosphorylation-dependent manner (see, e.g., Tyers et al., (2000) Curr Opin Genet Dev 10:54-64; Deshaies (1999) Annu Rev Cell Dev Biol 15:435-67; and Koepp et al., (1999) Cell 97:431-434). SCF consists of four subunits; three of which for a core complex (Skp1, Cdc53/Cul-1, Roc1) and the fourth, an F-box protein, confers substrate specificity of the SCF^{F-box} complex. Numerous F-box proteins are known in the art.

The present inventors determined that SCF modulates cyclin E turnover in yeast by comparing wild-type strain for cyclin E turnover with cdc53 and skp1 temperature sensitive (ts) mutants at the restrictive temperature. Cyclin E is stabilized in both the cdc53 and skp1 ts mutants, indicating that SCF activity is required for cyclin E turnover in yeast. The ubiquitin conjugating (E2) enzyme that usually works in concert with SCF in yeast is Cdc34. Cdc34 mutants are also found to stabilize cyclin E, further demonstrating that SCF ubiquitination modulates turnover of cyclin E in yeast.

Analysis of Cyclin E Turnover in Yeast

All yeast strains are isogenic to 15DaubΔ, a bar1 Δ ura3Δns, a derivative of BF264-15D. Several temperature sensitive skp1 mutants with different cell cycle arrest phenotypes are constructed by a combination of PCR mutagenesis and in vivo gap-repair similar to the procedure described by Muhlrad et al., (1992) Yeast 8:79-82. The skp1-24 mutant undergoes cell cyclin arrest with 1C DNA content and a multi-budded phenotype.

To analyze cyclin E turnover in various yeast mutants expressing cyclin E from an inducible GALL promoter, cells are grown in YEP-raffinose at 25° C. to an OD600=0.3. Cells are shifted to 35° C. for 30 min, then galactose is added to a final concentration of 2% to induce the GAL1 promoter. To terminate cyclin E expression after 60 min, cells are collected on filters and transferred to YEPD media and incubation is continued at 35° C. Extracts are prepared from aliquots taken after time periods of 0, 15, 45, 120, and 240 minutes and analyzed for cyclin E by Western blotting using monoclonal anti-cyclin E antibodies (HE12). Cells are lysed in RIPA buffer (1% deoxycholic acid, 1% Triton-X-100, 0.1% SDS, 250 mM NaCl, 50 mM Tris-HCl pH 7.5). A similar protocol is used to analyze the turnover of cyclin E phosphorylation site mutants in yeast except that a strain is used where human CDK2 is substituted for the endogenous Cdk Cdc2.

CDC4 Modulates Cyclin E Turnover in Yeasts

In yeast, the three best-characterized F-box proteins are Cdc4, Grr1, and Met 30. The present inventors measured the stability (turnover) of cyclin E in cdc4 and met30 mutants. It is determined that cyclin E turns over at the wild-type rate in a met30 mutant; however, cyclin E levels are stabilized in the cdc4 mutant (i.e., turnover is impaired or decreased). Thus, the inventors determined that, in yeast, cyclin E ubiquitination is mediated by the activity of the ubiquitin conjugating enzyme Cdc34 and the protein ubiquitin ligase, SCF^{Cdc}⁴. One caveat with this interpretation is that stabilization of the CDK inhibitor Sic1 in a cdc4 mutant might prevent cyclin E phosphorylation, thereby conferring stabilization indirectly. The inventors therefore investigated the stability of cyclin E in a cdc4/sic1 double mutant. It is determined that the turnover rate is unchanged relative to the cdc4 single mutant, confirming that Cdc4 is the F-box protein that couples SCF to cyclin E degradation in yeast, thereby modulating cyclin E degradation.

Phosphorylation of Cyclin E T62 and T380

The inventors discovered a second cyclin E phosphorylation site that acts synergistically with cyclin E T380-phospho to effect the degradation of cyclin E. Through analysis of the half-life of wild-type (wt) cyclin E in SCF mutants and a cyclin E T380A mutant (threonine to alanine substitution at threonine 380 of cyclin E) the inventors determined that the cyclin E T380A mutant is still susceptible to SCF-mediated ubiquitination and proteolysis. Cyclin E mutations were constructed at other potential phosphorylation sites. It was discovered that a T62A mutation also renders cyclin E more stable than wild-type. It was further discovered that a T62A/T380A mutation essentially eliminates ubiquitination of cyclin E and leads to a highly stable, low-turnover, cellular population of cyclin E.

The stability of wild-type and phospho-mutant cyclin E was determined as follows. Wild-type and phosphorylation site mutants of human cyclin E were expressed from the GAL1 promoter in a yeast cell where human CDK2 was substituted for the endogenous CDK. The stability/turnover of cyclin E was followed by immunoblotting using a specific anti-cyclin E antibody. The results are shown in Table 3.

TABLE 3
Relative Stability/Turnover of Cyclin E and
Phosphorylation Mutants Thereof

| | Chase (hours) | | | | | |
|---|---|---|---|---|---|---|
| Cyclin E type | 0 | 0.5 | 1 | 2 | 3 | 4 |
| wild-type | ++++ | Trace | − | − | − | − |
| T62A | ++++ | + | Trace | − | − | − |
| T380A | ++++ | ++ | + | Trace | − | − |
| T62A/T380A | ++++ | +++ | +++ | +++ | + | − |

Relative cyclin E content detected by immunoblotting with an antibody specific to human cyclin E. The yeast cells expressed wild-type, T62A phospho-mutant, T380A phospho-mutant, or T62A/T380 phospho-double mutant. GALL induced expression was discontinued at 0 hours. Cellular cyclin E content was determined at 0, 0.5, 1, 2, 3, and 4 hours. The “+” character indicates a unit of cyclin E content. “Trace” indicates that less than one unit of cyclin E content is observed at the particular time point. The “−” character indicates that cyclin E content was not detected at this time point by the present technique.

As shown in Table 3, wild-type cyclin E is degraded rapidly with a half-life of less than 0.5 hours. The general order of cyclin E stability is T62A/T380A>T380A>T62A>wild type from increasing stability to decreasing stability. The general order of cyclin E turnover is wild-type>T62A>T380A>T62A/T380A. The inventors contemplate that T62A/T380A turnover may occur through a non-specific protein degradation mechanism.

Identification of a Human HCDC4

The inventors identified a human expressed sequence tag (EST) in the NCBI database that, when translated, had significant homology to yeast Cdc4 (GenBank Accession BAA91986.1). Cdc4 contains seven evolutionarily conserved WD40 repeats, so many human proteins showed homology. However the protein defined by this EST, which the inventors designated hCdc4, exhibited significant homology outside of the WD40 repeat region, although to a lesser extent. Primers were derived from the hCdc4 EST sequence and the corresponding cDNA was obtained via RT-PCR. In vitro translation of this cDNA produced a polypeptide of approximately 63 kDa on SDS-PAGE. Further analysis of hCdc4 transcripts and genomic structure indicated that two alternative forms of the protein exist: α-hCdc4 and β-hCdc4.

The hCDC4 genomic locus was identified using the high throughput genomic sequence database (HTGS). The hCDC4 gene was found to be contained within BAC clones RP11 -555K12 (200147 bp, GenBank accession no. AC023424) and RP11-461L13 (208580 bp, GenBank accession no. AC080078). The World Wide Web tool NIX (Nucleotide Identify X), made available by UK HGMP Resource Centre, was used to aid the identification of several untranslated 5′ exons embedded within a predicted CpG island. RT-PCR was used to confirm all exon predictions.

The hCDC4 gene locus maps to chromosome region 4q32, which is frequently deleted in a broad spectrum of human tumor types and is comprised of 4 untranslated and 13 coding exons spanning approximately 210 Kb of the human genome (see FIG. 1A). RT-PCR was used to confirm exon order using HeLa cell mRNA as a template. Search of the EST databases combined with published reports revealed the existence of a third primary splice variant (designated γ-hCdc4). Ten common 3′ exons are alternatively spliced to three different 5′ coding exons. RT-PCR demonstrated that all 3 variants (a, p, and y) are expressed in HeLa cells, although the γ-form was difficult to amplify, suggesting a low abundance.

Northern blot analysis using probes specific for the various 5′ exons demonstrated that the α-splice variant of hCDC4 is expressed as a 5.5 Kb mRNA while the β- and γ-forms both are expressed as 4 Kb mRNAs. A CpG island is present 123 Kb upstream of the first coding exon of the α-form of hCdc4 and 4 small non-coding exons are differentially spliced to the α-coding exon. A search for proteins related to hCdc4 in other species suggested that homologues exist in D. melanogaster (dmCdc4) as well as C. elegans (Sel-10).

Plasmids Constructs

A human EST encoding part of the hCdc4 gene was amplified from HeLa mRNA by RT-PCR using two sequence-specific oligonucleotide primers, Pcr1 (gcaagcttatgggtttctacggcacatt (SEQ ID NO:42), forward), and Pcr2 (atgggccctgctcttcacttcatgtcc (SEQ ID NO:43), reverse), and TA-cloned into pCR2.1 (Invitrogen). The sequence of the cloned cDNA was verified in its entire length (1.7 Kb) by sequencing and found to match the sequence published in the NCBI database (GenBank accession no: BAA91986.1). The complete coding region corresponding to this cDNA was determined by 5′ RACE. Probing a Northern blot with sequences corresponding to specific exons, it was determined that this cDNA corresponds to a 4 kb hCdc4 mRNA expressed at high levels in only some cell types (e.g., brain and skeletal muscle). A second cDNA was identified by scanning upstream genomic sequences for probable exons and using these exons to probe a Northern blot. This cDNA was found to correspond to a ubiquitous 5.5 kb hCdc4 mRNA. Human cDNAs encoding the two full-length isoforms of the hCdc4 protein were amplified from HeLa mRNA by RT-PCR using sequence-specific oligonucleotide primers, Pcr4 (cttttggaaatgaatcaggaa (SEQ ID NO:44), forward) and Pcr2 (atgggccctgctcttcacttcatgtcc (SEQ ID NO:43), reverse) for the cDNA encoding the 110 kDa isoform, and Pcr5 (catgtatgtatgtgtgtcccg (SEQ ID NO:45), forward) and Pcr2 for the cDNA encoding the 69 kDa isoform, and subsequently TA-cloned into pCR2.1 (Invitrogen). The sequences of the cloned cDNAs were verified in their entire lengths (2.2 kb, and 1.9 kb, respectively) by sequencing. The cDNA encoding the 110 kDa isoform was deposited in GenBank (accession number: AY049984).

A mammalian transfection plasmid expressing N-terminal Flag-tagged hCdc4 protein was constructed by subcloning into pFLAG-CMV2 (Sigma) which provides the CMV2 promoter for high level expression. For expression in E. Coli, hCdc4 was tagged at the N-terminus with a RGS.His epitope through subcloning into pQE-10 (Qiagen).

The b-TrCP and Skp2 clones were gifts from Dr. F. Mercurio (Signal Pharmaceuticals, San Diego), and Dr. M. Pagano (Department of Pathology and Kaplan Comprehensive Cancer Center, New York University, New York), respectively, and were cloned into pFLAG-CMV2 to obtain b-TrCP and Skp2 tagged at their N-termini with the Flag-epitope. The mammalian transfection plasmid pCDNA3-Cul1-HA was a gift from Dr. R. Klausner (NIH, Bethesda, Md.), and pCDNA3-3MYCROC1 as well as pCDNA3-hSkp1 were gifts from Dr. Y. Xiong (Lineberger Cancer Center, University of North Carolina at Chapel Hill).

Baculovirus Constructs

Baculovirus expressing cyclin E with a GST-tag at its N-terminus was a gift from Dr. B. Sarcevic (The Garvan Institute, Sydney, Australia). Recombinant baculoviruses expressing GST-tagged versions of cyclin E phosphorylation site mutants (T62A, T380A, T62A/T380A) were generated using the pFastBac-system (Gibco BRL) according to the manufacturer's protocol. Baculovirus encoded proteins were expressed in SF9 insect cells grown in Ex-Cell 401 media (JRH) supplemented with 2% fetal bovine serum.

Adenoviral Constructs

DNAs encoding hCdc4 and an hCdc4 ΔF-box-mutant that had been deleted for its F-box using a two-step PCR protocol, as well as the cDNA coding for b-galactosidase were cloned into pDV46. Recombinant adenoviruses were generated by co-transfecting the recombinant plasmids and pBHG1021 into 293 cells using the calcium phosphate precipitation method.

hCDC4 Specifically Interacts with Cyclin E

To determine if hCdc4 interacts specifically with phosphorylated cyclin E, the 63 kDa and 110 kDa forms are independently incubated with glutathione beads bound to either free GST-cyclin E or GST-cyclin E/CDK2 complexes. In parallel samples, cyclin E was either phosphorylated or dephosphorylated. The hCdc4 was made by in vitro translation with incorporation of ³⁵S methionine to label the hCdc4. The ³⁵S-hCdc4 was visualized after PAGE.

In addition, in vitro translated ³⁵S-labeled b-TrCP and Skp2, were prepared and assayed for binding to either free or CDK2-bound GST-tagged cyclin E purified from SF9 insect cells on glutathione beads as a control of specificity between the hCdc4 and phosphorylated cyclin E. Both the free and CDK2-bound cyclin E were expressed and isolated from insect cells in their phosphorylated state (see example covering baculovirus expression). Dephosphorylation of cyclin E was performed after purification using λ phosphatase. The ³⁵S-labeled hCdc4 was also tested for binding to cyclin E that had been dephosphorylated followed by re-phosphorylation by its associated kinase, CDK2, in the presence of 1 mM ATP, and for binding to single phosphorylation site mutants (T62A, T380A) as well as the double mutant (T62A/T380A).

It was determined that hCdc4 binds to phosphorylated free or CDK2-bound cyclin E, but not to dephosphorylated cyclin E, regardless of CDK2 binding. Re-phosphorylation of cyclin E/CDK2 complexes following dephosphorylation restored binding. Thus hCdc4 binds specifically to phosphorylated cyclin E, either free or in complex with CDK2.

In control reactions, it was determined that in vitro translated b-TrCP, another human WD40-repeat-containing F-box protein did not bind to phosphorylated or dephosphorylated cyclin E. It is suggested in the literature that the human F-box protein Skp2, which contains leucine-rich repeats, targets cyclin E for ubiquitin-dependent degradation (see e.g., Yeh et al. (2001) Biochem Biophys Res Commun 281:884-890 and Nakayama et al. (2000) EMBO J 19:2069-2081). However, the present inventors discovered that in vitro translated Skp2 bound to neither phosphorylated nor dephosphorylated cyclin E, either free or bound to CDK2.

Accordingly, hCdc4 specifically interacts with phosphorylated cyclin E, does not specifically interact with dephosphorylated cyclin E, and regains specific interaction with a dephosphorylated cyclin E sample that is re-phosphorylated.

In Vitro Binding Assays

Complementary cDNAs encoding various hCdc4 isoforms and mutants, β-TrCP, and Skp2 were in vitro translated into [35S] methionine-labeled proteins using a T7 transcription/translation system (Promega). GST-tagged cyclin E and various cyclin E phosphorylation site mutants (including T62A, T380A, T62A/T380A, and other candidate phosphorylation site mutants) were expressed in baculovirus infected SF9 insect cells and adsorbed on glutathione beads for 1 hour at 4° C. after lysing the cells into GST-lysis buffer (50 mM HEPES-NaOH pH 7.5, 500 mM NaCl, 0.5% Tween-20, 1 mM EDTA, 1 mM EGTA, 1 mM DTT) supplemented with 5 mM sodium fluoride, 1 mM sodium orthovanadate, 10 mM b-glycerophosphate, 1 mM phenylmethyl sulfonylflouride, 2 mg/ml aprotinin, 2 mg/ml leupeptin, and 2 mg/ml pepstatin. After washing the beads first with lysis buffer and then with wash buffer (50 mM HEPES-NaOH pH 7.5, 0.01% Tween-20, 10% glycerol, 1 mM EDTA, 1 mM DTT) they were subjected to either phosphorylation reaction in kinase assay buffer (50 mM Tris-HCl pH 7.5,10 mM MgCl2) in the presence of 1 mM ATP or dephosphorylation reaction using A-phosphatase (NEB) for 1 hour at 30° C. The beads were washed three times with binding buffer (20 mM Tris-HCl pH 7.6, 200 mM NaCl, 0.5% NP-40, 1 mM EDTA, 1 mM DTT) prior to incubation with in vitro translated [35S] methionine-labeled proteins in 200 ml binding buffer for 2 hours at 4° C. Bound proteins were analyzed by SDS-PAGE followed by autoradiography.

Cell Culture and Immunological Techniques

A panel of breast cancer-derived cell lines were obtained from ATCC. and the University of Michigan Breast Cell/Tissue Bank and Database and grown in media recommended by the suppliers. HeLa, Kb (human epidermoid carcinoma), and 293T cells were grown in DMEM (Gibco BRL) supplemented with 10% fetal bovine serum. All cells were maintained in a humidified 37° C. incubator with 5% CO₂. 293T cells were transfected with various combinations of plasmids in 10 cm-dishes using the calcium phosphate precipitation method. Forty hours post-transfection, cells were lysed, as indicated, into either mammalian cell lysis buffer 1, MCLB1 (20 mM Tris-HCl pH 7.5, 150 mM NaCl, 0.5% NP-40, 1 mM EDTA, 1 mM EGTA, 1 mM DTT) or mammalian cell lysis buffer 2, MCLB2 (20 mM Tris-HCl pH 7.5,150 mM NaCl, 0.25% NP-40, 10% glycerol, 1 mM EDTA, 1 mM DTT), supplemented with 5 mM sodium fluoride, 1 mM sodium orthovanadate, 1 mM phenylmethyl sulfonylflouride, 2 mg/ml aprotinin, 2 mg/ml leupeptin, and 2 mg/ml pepstatin. After centrifugation at 4° C. (14.000 rpm, 15 min) lysates (1 mg/0.5 ml) were subjected to immunoprecipitation using 10 mg anti-Flag antibody immobilized on agarose beads (M2, Sigma) for 2 hours at 4° C. Immune complexes were washed three times with lysis buffer and subsequently used for ubiquitination reactions or analyzed by immunoblotting. Polyclonal antibodies against human Cdc4 were generated in rabbits after injection of recombinant Cdc4 protein produced in bacteria (E. coli). Antibodies were affinity-purified using nitrocellulose-bound antigen. Commercially obtained antibodies used in this study: Mouse monoclonal anti-Flag antibody (M2, Sigma), mouse monoclonal anti-HA antibody (HA.11, Babco), rabbit polyclonal anti-Myc antibody (A-14, Santa Cruz), rabbit polyclonal anti-Cdk2 antibody (M2, Santa Cruz), and rabbit polyclonal antibodies against Cull, Skpl, and ROC1 (Neomarkers).

hCDC4 and Cyclin E Phosphorylation Status

In this example, the inventors demonstrate relative contributions of the T62 and T380 phosphorylation sites to hCdc4 binding specificity. The specificity of hCdc4 for phosphorylated and dephosphorylated cyclin E site mutants: T380A, T62A, and a T62A/T380A double mutant was assayed as described in the previous example for wild-type cyclin E. Observations of specific binding of hCdc4 (63 kDa) to each cyclin E type are set forth in Table 4 below.

TABLE 4
Specificity of hCdc4 and Cyclin E Interaction

| | Specific binding with hCdc4 | |
|---|---|---|
| Cyclin E phosphorylation status | Phosphorylated | Dephosphorylated |
| Cyclin E wild-type | ++++ | − |
| Cyclin E T380A | ++ | − |
| Cyclin E T62A | +++ | − |
| Cyclin E T62A/T380A | − | − |

Table 4 shows the relative specific binding of hCdc4 with cyclin E types listed on the left, wherein the cyclin E is phosphorylated or dephosphorylated as indicated. Each “+” character indicates a unit of relative specificity of the interaction. The “−” character indicates lack of a specific interaction.

The data in Table 4 indicate that the interaction between hCdc4 and phosphorylated wild-type cyclin E is highly specific. The results observed using cyclin E T62A indicate that the cyclin E T380 phosphorylation site is of primary importance to the specificity of interaction with hCdc4. The results observed using cyclin E T380 A indicate that the cyclin E T62 phosphorylation site is of secondary importance to the instant specificity. No interaction, or non-specific interaction, is observed between hCdc4 and the cyclin E T62A/T380A double mutant. This demonstrates that the cyclin E T62 and T380 phosphorylation sites can account for essentially all specific binding activity with hCdc4. It also demonstrates that phosphorylation the T62 and T380 sites of cyclin E act synergistically to determine binding specificity with hCdc4.

In Vitro Modulation of Cyclin E Turnover by HCDC4

To determine whether expression of hCdc4 in vivo has an impact on cyclin E turnover, a recombinant adenovirus was constructed containing the hCdc4 cDNA under control of the CMV promoter. When Kb cells were transduced with the hCdc4 adenovirus, the endogenous cyclin E levels were dramatically reduced compared to control adenovirus transductions (see Table 5 below).

TABLE 5
In Vivo Modulation of Cyclin E Turnover by hCdc4

| | Adenovirus | | | | |
|---|---|---|---|---|---|
| | hCdc4 | β-Gal | ΔF-box-hCdc4 | control | Antibody |
| Cyclin E | + | +(P)/++++ | ++++(P)/++ | +(P)/++++ | α-cyclin |
| | | | | | E 1B |
| hCdc4 | 110/69 | − | ++++ | − | α-hCdc4 |
| | kDa | | | | 1B |
| | (++/++) | | | | |
| CDK2 | ++++ | ++++ | ++++ | ++++ | α-CDK2 |
| | | | | | 1B |

The “+” character indicates a relative amount of the particular polypeptide as indicated in the column at left (detected by a specific antibody indicated in the column at right). The “(P)” indicates that the polypeptide had a reduced SDS-PAGE mobility indicating phosphorylation. The “−” character indicates no significant bands observed.

Expression of α-hCdc4 by the hCdc4 adenovirus vector resulted in the production of the 110 kDa and 69 kDa isoforms of hCdc4 in the Kb cells (the doublet at 110 kDa and 69 kDa detected by the α-hCdc4 specific antibody). As expected, hCdc4 is not expressed by a β-Gal adenoviral vector or an empty vector (no insert included).

The data in Table 5 demonstrate that administration of an hCdc4 adenovirus vector, preferably an α-hCdc4 adenovirus vector, substantially enhances cellular turnover of cyclin E in vivo (e.g., by 2 fold or more). Conversely, administering an adenovirus expressing an F-box-deleted hCdc4, and thereby a dominant negative hCdc4 allele, substantially blocks cellular degradation of cyclin E in vivo (e.g., a 2 fold or more accumulation of cyclin E above control). In addition, the bulk of the accumulated cyclin E in the Δ-F-box hCdc4 expressing cells was hyper-phosphorylated based on a reduced SDS-PAGE mobility.

Further, ³⁵S-methionine pulse-chase experiments were performed on parallel adenoviral transductions. Consistent with the observed changes in cyclin E steady-state level resulting from alterations in the rate of ubiquitin-mediated proteolysis, transduction of wild-type hCdc4 led to a decrease in cyclin E half-life whereas transduction of the Δ-F-box hCdc4 (dominant negative) led to an increase in the cyclin E half-life. Thus, the inventors contemplate that hCdc4 levels are rate limiting for cyclin E turnover. To confirm that the F-box-deleted version of hCdc4 indeed had the appropriate characteristics to behave as a dominant negative, the inventors showed by immunoprecipitation that it binds specifically to phosphorylated cyclin E in vivo but not to components of SCF (Cul1, ROC1, Skp1). The inventors also showed that in vitro translated ΔF-box-hCdc4 binds specifically to phosphorylated cyclin E.

Wild-Type hCDC4 Interacts with an SCF Complex

This example demonstrates that wild-type hCdc4 is part of an SCF complex. Flag-tagged hCdc4 was introduced into 293T cells by transfection. Anti-Flag immunoprecipitates were then probed for SCF components by SDS-PAGE followed by Western blotting. Based on co-immunoprecipitation, hCdc4 is associated with both endogenous and co-transfected core components of human SCF: Skp1, Cul-1, and Roc1.

SCF^{hCDC4} Modulates Cyclin E Ubiquitination

This example demonstrates that SCF^{hCdc4} ubiquitinates cyclin E in a phosphorylation dependent manner (i.e., dependent on phosphorylation of cyclin E). Expression plasmids for Flag-hCdc4, as well as the other three components of SCF (Skp1, Cul1 and Roc1) were co-transfected into 293T cells. Anti-Flag immunoprecipitates were then tested for ability to ubiquitinate phosphorylated cyclin E. Immunoprecipitated SCF^{hCdc4} was capable of specifically and efficiently ubiquitinating phosphorylated cyclin E, either free or bound to CDK2.

In this experiment there was some ubiquitination of dephosphorylated cyclin E, but this is most contemplated to result from re-phosphorylated of the cyclin E in the ubiquitination reaction due to the presence of an ATP-regenerating system, even in the presence of the CDK2 competitive inhibitor roscovitine, which was included in the reaction mixture.

To confirm that the slowly migrating derivatives of cyclin E observed were indeed poly-ubiquitinated products, increasing amounts of chain-terminating methylated ubiquitin were added to parallel reactions. Addition of methylated ubiquitin increased the mobility of the cyclin E derivatives confirming that the modification is indeed ubiquitination.

In parallel experiments using Flag-tagged β-TrCP and Skp2, respectively, anti-Flag immunoprecipitates were incapable of efficiently ubiquitinating either phosphorylated or dephosphorylated cyclin E even though SCF complexes were formed (i.e., SCF^{β-TrcP} and SCF^{Skp2}, respectively). On the other hand, immunoprecipitated SCFSkp2 specifically ubiquitinates phosphorylated p27 ^{Kip1}, one of its established targets.

Consistent with the examples described above, SCF^{hCdc4} mediated ubiquitination of cyclin E (T62A) was slightly reduced relative to wild-type, ubiquitination of cyclin E (T380A) was moderately reduced, and the double mutant was not ubiquitinated or essentially not ubiquitinated. Thus, hCdc4 is incorporated into an SCF complex, SCF^{hCdc4}, that efficiently ubiquitinates phosphorylated but not unphosphorylated cyclin E. Furthermore, transduced wild-type and dominant negative hCdc4 dramatically effect the steady-state levels of cyclin E in vivo. Accordingly, the inventors set forth that SCFhCdc4 is the predominant pathway mediating cyclin E specific turnover in mammalian cells.

In Vitro Ubiquitination Assay

Recombinant SCF complexes containing different Flag-tagged F-box proteins were isolated, using anti-Flag antibodies immobilized on agarose beads, from transfected 293T cells lysed into mammalian cell lysis buffer 2 supplemented with 5 mM sodium fluoride, 1 mM sodium orthovanadate, 1 mM phenylmethyl sulfonylflouride, 2 mg/ml aprotinin, 2 mg/ml leupeptin, and 2 mg/ml pepstatin. Individual immune complexes were washed three times with lysis buffer and three times with ubiquitination reaction buffer (20 mM Tris-HCl pH 7.4, 5 mM MgCl2, 1 mM DTT). GST-tagged cyclin E and cyclin E phosphorylation site mutants purified from baculovirus infected SF9 insect cells on glutathione beads were subjected to either phosphorylation or dephosphorylation followed by elution of the bound proteins with elution buffer (50 mM HEPES-NaOH pH 7.5, 0.01% Tween-20, 10% glycerol, 1 mM EDTA, 1 mM DTT) containing 15 mM reduced glutathione. Eluates from multiple elution steps were pooled and concentrated on Centricon-30 spin columns (Amicon) and stored in aliquots at −80° C. Equal amounts of SCF immune complexes were mixed with different eluted cyclin E proteins for 30 min on ice to allow binding. Subsequently, aliquots of this mixture were added to ubiquitination reactions in a total volume of 30 ml containing 15 mg of bovine ubiquitin (Sigma), 0.5 mg of yeast E1 enzyme (Boston Biochem), 1 mg of human 6xHis-Cdc34 purified from bacteria, and an ATP-regenerating system (1 mM ATP, 20 mM creatine phosphate, 0.1 mg/ml creatine kinase) in ubiquitination reaction buffer supplemented with 5 mM sodium fluoride, 1 mM sodium orthovanadate, 1 mM phenylmethyl sulfonylflouride, 2 mg/ml aprotinin, 2 mg/ml leupeptin, and 2 mg/ml pepstatin. The CDK2-inhibitor roscovitine (Biomol; 100 mM final concentration) was added to reactions containing dephosphorylated cyclin E as substrate. Reactions were incubated at 30° C. for 2 hours, terminated by boiling for 5 min with SDS-sample buffer, and analyzed by SDS-PAGE followed by immunoblotting using anti-cyclin E antibodies. The ubiquitination assay using p27 as a substrate was performed as described in Spruck et al. (2001) Mol. Cell 7:639-650.

Expression Patterns of hCDC4 in Mammalian Tissues

Using Northern blot techniques of RNA extracted from various tissues, the inventors determined that hCdc4 is expressed in most, if not all, tissues including, but not limited to: brain, heart, skeletal muscle, colon, thymus, spleen, kidney, liver, small intestine, placenta, lung and peripheral blood lymphocytes. In most tissues, the predominant mRNA species is approximately 5.5 kb in length. Certain tissues expressed a 4 kb mRNA as the predominant form, specifically in brain and skeletal muscle tissues. These tissues also exhibited the highest level of expression of hCdc4 and expressed both the 5.5 kb and 4 kb mRNA species. The fact that hCdc4 is expressed at high levels in non-proliferating tissues (brain and skeletal muscle) suggests a function in addition to turnover of cyclin E, since cyclin E expression should be limited to tissues undergoing cell division. These data also suggest that the 5.5 kb mRNA, which is ubiquitously expressed, encodes the F-box protein physiologically responsible for specifically targeting cyclin E for ubiquitination and degradation. It is contemplated by the inventors that the 4 kb mRNA isoform, which is expressed predominantly in essentially non-proliferating tissues, encodes a hCdc4 polypeptide isoform with a physiological role other than specifically targeting cyclin E for ubiquitination and degradation.

Using exon-specific probes, the inventors demonstrate that the 5.5 kb mRNA encodes the 110 kDa α-hCdc4 isoform whereas the 4 kb band includes two hCdc4 encoding isoforms, one which encodes the β-hCdc4 polypeptide and a second that encodes the γ-hCdc4 isoform. Analysis of hCdc4 expression in synchronized HeLa cells demonstrated that neither hCdc4 mRNA nor hCdc4 protein is cell cycle regulated.

Pulse-Chase Northern and Southern Analysis

Pulse-chase experiments were performed on thymidine arrested cells as described in Spruck et al. (2001) Mol. Cell 7:639-650. Thymidine arrested Kb cells were co-transduced with adenovirus encoding cyclin E (resulting in a 5-10 fold elevation over endogenous levels) and virus encoding either wild-type hCdc4, an F-box deleted hCdc4, or a control β-galactosidase. Viral transductions were incubated for 24 hours prior to pulse-chase. Immunoprecipitations were performed with a monoclonal anti-cyclin E antibody (HE172). Quantitation of bands was performed using ImageQuant software (Molecular Dynamics). For Northern blot analysis, 2 mg of poly[A]+ RNA was isolated from asynchronously growing cultures according to the manufacturer's protocol (Qiagen) and run on a 1% formaldehyde agarose gel as described25. The gel was blotted onto Zeta-Probe GT genomic membrane (Bio-Rad) and hybridized with a radiolabeled hCdc4 probe followed by autoradiography. For Southern blot analysis, 10:g of DNA was digested with Sstl and EcoRV, run on 0.8% agarose gels, blotted and probed with the cDNA encoding the 69 kDa form of hCdc4.

Loss of hCDC4

The present example demonstrates that loss of hCdc4 activity is causative for elevated cyclin E protein levels and is associated with tumorigenesis. Loss of hCdc4 activity includes aberrant hCdc4 mRNA expression, a decrease or loss of in hCdc4 protein levels, a defect in the hCdc4 genetic locus. Mutations in hCdc4 can inhibit interaction between the hCdc4 and phosphorylated cyclin E and/or the SCF complex, for example. This example further discloses a method for identifying cells with defects in hCdc4.

Eight randomly chosen breast cancer-derived cell lines and a breast epithelial cell line (184A1, normal control) were analyzed for cyclin E expression by immunoblotting, and for hCdc4 transcripts by Northern blot analysis (throughout this example, refer to Table 6, below). Two of the breast cancer-derived cell lines, MDA-MB-157 and SUM149PT, exhibited a significant elevation of cyclin E above the level observed in the control 184A1 cells, an immortalized, non-transformed breast epithelial cell line. One of the high cyclin E-expressing cell lines was shown previously to contain a genomic amplification of the cyclin E locus (MDA-MB-157, see Keyomarsi et al. (1993) Proc Natl Acad Sci U.S.A. 90:1112-1116).

In order to analyze the hCdc4 mRNA expression status in these cell lines, polyA+ mRNA was prepared and subjected to Northern blot analysis using the hCdc4 cDNA as a probe. The 184A1 breast epithelial cell line contained a predominant hybridizing mRNA species at approximately 5.5 kb and a lower abundance species at approximately 4 kb. These correspond to the two species observed in the tissue blot. Most of the breast carcinoma-derived cell lines expressed only the 5.5 kb species. However, one cell line, SUM149PT, that exhibited elevated cyclin E levels expressed an mRNA species of reduced mobility. The lack of any of the mRNA species characteristic of hCdc4 suggests a mutational lesion and, furthermore, loss of heterozygosity (LOH).

In order to determine whether the observance of the aberrant mRNA species in the SUM149PT cell line corresponds to a loss or alteration of hCdc4 protein, antibodies were raised against hCdc4 produced in E. coli and shown to react specifically with hCdc4 in crude extracts of cells by Western blotting, wherein the cell were transfected with an hCdc4 expressing vector. The hCdc4 specific antibodies were used to detect hCdc4 protein in crude extracts of non-transfected cells by concentrating the hCdc4 in the extracts either by adsorption to immobilized phosphorylated cyclin E or immunoprecipitation using anti-hCdc4 antibody and then, in each case, subjecting the samples to SDS-PAGE and Western blotting with anti-hCdc4 antibody. The 184A1 cells and a breast carcinoma-derived cell line that does not exhibit elevated cyclin E levels (T47D) contain the 110 kDa hCdc4 isoform (the α-hCdc4 isoform) detected either by binding specifically to phosphorylated cyclin E or by immunoprecipitation using anti-hCdc4 antibody. HeLa cells also express this species.

However, SUM149PT expresses no hCdc4 cross-reactive protein capable of being immunoprecipitated or binding to phosphorylated cyclin E within the limit of detection, consistent with the interpretation that the mutation responsible for producing the aberrant mRNA also eliminates or severely reduces expression of hCdc4, resulting in elevated levels of cyclin E. The breast epithelial cell line 184A1 and the breast cancer-derived cell lines SUM149PT, and T47D were analyzed for expression of hCdc4 protein by anti-hCdc4 immunoprecipitation followed by immunoblotting using specific anti-hCdc4 antibodies, or incubating crude lysates prepared from these cell lines with either dephosphorylated (control) or phosphorylated GST-cyclin E immobilized on glutathione beads followed by SDS-PAGE and Western blotting using anti-hCdc4 antibodies. An intense band migrating ahead of hCdc4 in the immunoprecipitation-immunoblot experiment corresponds to IgG heavy chain-light chain heterodimers.

In addition, ³⁵S-methionine pulse-chase experiments support this interpretation in that cyclin E has an extended half-life in the SUM149PT cell line relative to 184A1 and T47D. The ³⁵S-methionine pulse-chase analysis was performed to measure the turnover rate of cyclin E in the 184A1, SUM149PT, and T47D cell lines arrested in S-phase with 1.0 mM thymidine.

In order to determine the nature of the mutation at the hCdc4 locus in the SUM149PT cell line, the presumptive protein coding region was amplified by RT-PCR. Sequencing of the amplified cDNA revealed it to contain a direct repeat of exons 8 and 9 separated by 11 base pairs of intronic sequence. This mutation would be predicted to result in chain termination, eliminating the last 4 (out of 7) WD40 repeats, presumably rendering the resulting polypeptide non-functional. The aberrant hCdc4 mRNA from SUM149PT is approximately 11 kb in length.

To test the activity of this mutant hCdc4, in vitro translation of the cDNA isolated from the SUM149PT cell line was conduced and produced a truncated product that did not bind to phosphorylated cyclin E. The loss of heterozygosity and internal genomic duplication at the hCdc4 locus was confirmed by Southern blotting. This finding and the causative role of elevated cyclin E in carcinogenesis suggests that hCdc4 is a tumor suppressor associated with certain types of malignancy (e.g., malignancies with an elevated level of cyclin E protein), including breast cancers.

TABLE 6
Defects in hCdc4 Lead to Elevated Cyclin E Levels and
Tumorigenesis

| Cell Line | 184A1 | MDA-MB-435S | T47D | BT-549 | ZR-75-1 |
|---|---|---|---|---|---|
| Cyclin E protein | + | + | + | + | + |
| Approx. t_{1/2} | <1 hr | | <1 | | |
| | | | hr | | |
| hCdc4 mRNA | 5.5 > 4 | 5.5 > 4 | 5.5 > | 5.5 > 4 | 5.5 > 4 |
| | | | 4 | | |
| hCdc4 protein | | | + | | |
| hCdc4 binds P | Yes | | Yes | | |
| hCdc4 locus | | | | | |
| Description | Control | | | | |

TABLE 6 (CONTINUED)

| Cell Line | MDA-MB-436 | MDA-MB-157 | SUM149PT | SK-BR-3 |
|---|---|---|---|---|
| Cyclin E protein | + | ++ | ++++ | + |
| Approx. t_{1/2} | | | >4 hrs | |
| hCdc4 mRNA | 5.5 > 4 | 5.5 > 4 | loss of 5.5 and 4, | 5.5 > 4 |
| | | | gain of 11 kb | |
| hCdc4 protein | | | Absent | |
| hCdc4 binds P | | | Absent/No | |
| hCdc4 locus | | | Defect with loss of | |
| | | | heterogeneity | |
| Description | | Amplification of | Loss/defect | |
| | | cyclin E gene | of hCdc4 | |

Referring to Table 6, the “+” character means that cyclin E was present in the samples. The “++” character indicates that cyclin E was present in elevated abundance in the samples (approximately two-fold elevation over cyclin E protein levels observed in the control cell line 184A1). The “++++” character indicates that cyclin E was present in highly elevated abundance in the samples (four-fold elevation or more over cyclin E protein levels observed in the control cell line 184A1). The phrase “hCdc4 binds P” means that the hCdc4 protein specifically binds phosphorylated cyclin E.

Defective hCDC4 in Endometrial Adenocarcinoma

This example demonstrates a correspondence between defective hCdc4 expression/activity and endometrial adenocarcinoma. The inventors analyzed samples of human endometrial adenocarcinomas for alterations in the hCDC4 gene, mRNA and the hCdc4 protein. Previous reports indicate that elevated cyclin E levels occur frequently in endometrial adenocarcinomas (see, e.g., Milde-Langosch et al. (2001) J Cancer Res Clin Oncol 127:537-544). Defects in hCdc4 were identified in approximately 16% or more of the endometrial adenocarcinomas. Defects in hCdc4 were identified in approximately 50% or more of tumors with either elevated cyclin E protein levels or elevated phosphorylated cyclin E levels. Defects in hCdc4 were identified in approximately 85% or more of tumors with an elevation of phosphorylated cyclin E.

Western blot analysis on 51 frozen tumor specimens demonstrated that 8 contained elevated levels of cyclin E protein (see Table 7, tumor numbers 1-8). In two of these tumors the elevated cyclin E phenotype is contemplated be result from genomic amplification of the cyclin E locus. The inventors further discovered a specific accumulation of phosphorylated cyclin E in 7 tumors (see Table 7, tumors 7-13). This assessment was based on low mobility on SDS-PAGE and confirmed by phosphatase treatment. Surprisingly, five tumors (Table 7, tumors 9-13) contained only low to moderate levels of cyclin E protein (i.e., normal); but displayed an accumulation of phosphorylated cyclin E.

These data demonstrate a correspondence between high grade tumors, with a generally poor prognosis, and an accumulation of phosphorylated cyclin E. For example, tumors 9-13 showed an accumulation of phosphorylated cyclin E and a corresponding average tumor grade of 3 (high grade). Tumors 14-51, on the other hand do not display increased cyclin E (phosphorylated or non-phosphorylated) and have an average tumor grade of 1.4 (low grade). Accordingly, an elevated phosphorylated cyclin E content is a marker or endometrial adenocarcinoma. In addition, an elevated phosphorylated cyclin E content is a prognostic marker for endometrial adenocarcinoma, indicating a poor prognosis.

TABLE 7
Analysis of Endometrial Adenocarcinomas

| Tumor | | | | hCDC4 | Lymph |
|---|---|---|---|---|---|
| No. | Grade/Stage | cyclin E | Phos-cyclin E | mutation | node 1 |
| 1 | G2/1.3 | + | − | − | n/a |
| 2 | G3/3.1 | + | − | − | − |
| 3 | G3/1.3 | + | − | − | − |
| 4 | G3/4.2 | + | − | − | n/a |
| 5 | G3/2.2 | + | − | − | n/a |
| 6 | G3/3.3 | + | − | + | + |
| 7 | G3/3.3 | + | + | + | + |
| 8 | G3/1.3 | + | + | + | n/a |
| 9 | G2/3.3 | − | + | + | + |
| 10 | G3/1.3 | − | + | + | n/a |
| 11 | G3/1.3 | − | + | + | n/a |
| 12 | G3/n/a | − | + | + | n/a |
| 13 | G3/2.1 | − | + | − | − |
| 14-51 | Ave. Grade 1.4 | − | − | 1/15 | 4/13 |
| | Ave. Stage 2.4 | | | | |

Table 7 provides data from an analysis of 51 endometrial adenocarcinomas. Tumors 1-13 in this listing included a higher than normal cyclin E protein content, an accumulation of phosphorylated cyclin E, or both. Tumors 14-51 had a normal cyclin E and phospho-cyclin E profile. The tumor grade is on a scale of 1, 2, 3, or 4. The tumor grade corresponds to the relative differentiation of cells of the tumor. In general, grade 1 (G1) is a well differentiated tumor (low grade), G2 is a moderately well-differentiated tumor (intermediate grade), G3 is a poorly differentiated tumor (high grade), and G4 is an undifferentiated tumor (high grade). The aggressiveness of the tumor increases with increasing grade. The prognosis of a patient with a tumor, generally, worsens with increasing tumor grade. In general, increasing tumor stage is a relative measure of the extent of a cancer within the body. For example, a stage 1 tumor is typically localized; a stage 2 tumor has typically spread to a nearby, usually proximal tissue; a stage 3 tumor has typically spread to a nearby, but not necessarily proximal tissue; and a stage 4 tumor has typically spread within a region, numerous tissues, or distant parts of the body. A “+” character means that the indicated molecule is present above normal or background levels. A “−” character means that the indicated molecule is not detected or present at normal or background levels.

Single-strand conformation polymorphism (SSCP) analysis of the hCDC4 gene for 1) tumors that exhibited either elevated cyclin E levels or phosphorylated cyclin E or both (13/51) and 2) fifteen control tumors that had neither elevated cyclin E levels nor phosphorylated cyclin E. Aberrant SSCP banding patterns, indicative of mutations, were observed in 8 tumors (see Tables 8 and 9). Interestingly, 6 of 8 tumors with hCDC4 gene mutations also accumulated phosphorylated cyclin E and only 2 of these tumors contained elevated cyclin E protein levels (see Table 7). A wild-type SSCP banding pattern was observed for 14/15 control tumors (with neither elevated nor phosphorylated cyclin E). DNA sequencing demonstrated that 6/8 mutations occurred within the 7 WD40 repeat domains of hCdc4 that are proposed to be involved in substrate recognition (Table 9). Four mutations introduced a stop codon within the WD40 repeat region. Furthermore, two missense mutations occurred at Arg residues that are conserved in the Cdc4 homologs of S. cerevisiae, Drosophila, C. elegans and human. One mutation (Glu->Tyr, codon 124) occurred outside the WD40 repeat regions, in the 5′ exon of α-hCDC4. Another mutation was localized to the 5′ exon of the β-hCdc4 (GTT->ATT, codon 23). This mutation was obtained from one of the “control” tumors that had neither elevated nor phosphorylated cyclin E. Of the 8 hCDC4 gene mutations detected, the 6 that were localized to the WD40 repeat region, and therefore presumably prevented substrate binding, occurred in tumors with an accumulation of phosphorylated cyclin E. In contrast, the two tumors that contained hCDC4 gene mutations that were localized to the amino terminal region of hCdc4, contemplated to not affect substrate binding, did not accumulate phosphorylated cyclin E. Thus, it is contemplated that the accumulation of phosphorylated cyclin E may depend on an inability of hCdc4 to bind substrate.

Only those tumors showing elevated cyclin E levels or increased cyclin E phosphorylation and 15 control tumors were tested for hCdc4 mutations by SSCP. Accordingly, it is contemplated that the 8 hCdc4 mutations identified in 51 endometrial adenocarcinomas (16%) is contemplated to be a minimal estimate of the correspondence of hCdc4 mutation with formation of endometrial adenocarcinoma. No mutations in hCdc4 were detected in paired normal tissue DNA corresponding to any tumor, confirming that the hCDC4 gene mutations in the tumors were of somatic origin.

TABLE 8

| | | No. Tumors | hCDC4 Gene |
|---|---|---|---|
| | No. Tumors | Per Grade | Mutation |
| Elevated Cyclin E | 6 | 5 (G3) 1 (G2) | 1/6 |
| Protein (only) | | | |
| Phospho-Cyclin E | 5 | 4 (G3) 1 (G2) | 4/5 |
| Protein (only) | | | |
| Elevated Cyclin E | 2 | 2 (G3) | 2/2 |
| and | | | |
| Phospho-Cyclin E | | | |
| Protein | | | |
| Neither | 38 | 16 (G3) 22 (G2) | 1/15 |

TABLE 9

| Tumor | | | | LOH |
|---|---|---|---|---|
| No. | Mutation | Codon | Result | 4q32 |
| 6 | GAG→TAT | 124 | Glu→Tyr | + |
| 7 | AAA→(TC)A (2 bp insertion) | 371 | Lys→Ser, Term codon 376 | + |
| 8 | CGA→TGA | 367 | Arg→Ter | + |
| 9 | CGT→CAT | 465 | Arg→His | ni3 |
| 10 | CGA→CAA | 479 | Arg→Gln | + |
| 11 | CGA→TGA | 658 | Arg→Ter | + |
| 12 | AGA→A(A)G (1 bp insertion) | 472 | Arg→Lys, Term. codon 476 | + |
| 14 | GTT→ATT | 23 | Val→Ile | + |

Referring to Table 9, “Tumor No.” refers to the number assigned to each of the 51 tumors being analyzed; “Codon” refers to the codon number based on α-hCdc4 polynucleotide (e.g., SEQ ID NO:1); the mutation in tumor number 14 is in the β-exon 1 of the β-hCdc4 isoform at codon 23; “ni” means non-informative at all loci analyzed. Each of tumors 7, 8, 9, 10, 11, and 12 include a mutation resulting in a polypeptide variation in the C-terminal common domain of hCdc4. The mutation in tumor number 6 is in the α-exon 1 of the α-hCdc4 encoded isoform.

The inventors contemplate that a mutation in the C-terminal common domain of hCdc4 (e.g., SEQ ID NO:14) or, optionally, a mutation that inhibits substrate interaction, results in 1) decreased turnover of phosphorylated cyclin E; 2) accumulation or elevation of phosphorylated cyclin E; and 3) tumorigenesis. Substrate interaction preferably refers to binding of the hCdc4 with phosphorylated cyclin E and/or SCF.

The SSCP data suggested that most of the tumors that contained hCDC4 gene mutations did not retain a wild-type allele of hCDC4, as indicated by the absence of a wild-type banding pattern. These tumors were examined further for LOH of several markers surrounding the hCDC4 gene on chromosome region 4q32. Evidence of LOH was observed in 7 cases where informative heterozygosity was apparent in matched non-tumor samples, confirming the loss of the remaining hCDC4 allele. A single tumor was non-informative at all loci analyzed (see Table 9). These data suggest that hCdc4 is a tumor suppressor and loss of hCdc4 leads to a variety of tumors including those observed in 4q32 region.

### REFERENCES

All patents, publications, articles, and other citations set forth herein (including the following), are hereby incorporated by reference in their entirety in the specification of the present application.

U.S. Pat. No. 5,710,001 to Skolnick et al.

U.S. Pat. No. 5,807,746 to Lin et al.

U.S. Pat. No. 6,312,956 to Lane.

U.S. Pat. No. 6,331,284 to Hung et al.

U.S. Pat. No. 6,417,429 to Hein et al.

Bett et al. (1994) Proc Natl Acad Sci USA 91:8802-8806.

Bortner et al. (1997) Mol Cell Biol 17:453-459.

Clurman et al. (1996) Genes Dev 10:1979-1990.

Dealy et al. (1999) Nat Genet 23:245-248.

Deshaies et al. (1999) Annu Rev Cell Dev Biol 15:435-467.

Donnellan et al. (1999) Faseb J 13:773-780.

Ekholm et al. (2000) Curr Opin Cell Biol 12:676-684.

Ekholm et al. (2001) Mol Cell Biol 21:3256-3265.

Erlanson et al. (2001) Leuk Lymphoma 40:461-470.

Gupta et al. (2001) J Biol Chem 276:34371-34378.

Hayashi et al (2001) Lung Cancer 34:59-65.

Keyomarsi et al. (1993) Proc Natl Acad Sci USA 90:1112-1116.

Keyomarsi et al. (1995) Oncogene 11:941-950.

Knudson JAMA 241:279.

Knuutila et al. (1999) Am J Pathol 155:683-694.

Koepp et al. (1999) Cell 97:431-434.

Koepp et al. (2001) Science 294:173-177.

Li et al. (2001) Hum Pathol 32:945-953.

Milde-Langosch et al. (2001) J Cancer Res Clin Oncol 127:537-544.

Moberg et al. (2001) Nature 413:311-316.

Muhirad et al. (1992) Yeast 8:79-82.

Nakayama et al. (2000) Embo J 19:2069-2081.

Patton et al. (1998) Trends Genet 14:236-243.

Porter et al. (1997) Nat Med 3:222-225.

Reed et al. (1985) Proc Natl Acad Sci USA 82:4055-4059.

Reed Biochim Biophys Acta (1996) 1287:151-153.

Reed Cancer Surv (1997) 29:7-23.

Sambrook et al. 1989) Molecular Cloning: A laboratory manual; Cold Spring Harbor

Laboratory, Cold Spring Harbor Publishers.

Sandhu et al. (2000) Cancer Detect Prev 24:107-118.

Sauer et al., Prog Cell Cycle Res (1995) 1:125-139.

Singer et al. (1999) Genes Dev 13:2375-2587.

Spruck et al. (1999) Nature 401:297-300.

Spruck et al. (2001) Mol. Cell 7:639-650.

Strohmaier et al (2001) Nature 413:316-322.

Tyers et al (200 o) Curr Opin Genet Dev 10:54-64.

Walen et al. (1989) Cancer Genet Cytogenet 37:249-261.

Wang et al. (1996) Curr Biol 9:1191-1194.

Won et al. (1996) EMBO J 15:4182-4193.

Wu, G. et al. (2001) Mol Cell Biol 21:7403-7415.

Yeh et al. (2001) Biochem Biophys Res Commun 281:884-890.

What is claimed is:

1) A composition comprising a purified polypeptide including an amino acid sequence set forth in SEQ ID NO:4.

2) The composition of claim 1, wherein the polypeptide consists essentially of the amino acid sequence set forth in SEQ ID NO:4.

3) The composition of claim 1, wherein the polypeptide includes the amino acid sequence set forth in SEQ ID NO:2.

4) The composition of claim 1, wherein the polypeptide consists essentially of the amino acid sequence set forth in SEQ ID NO:2.

5) A purified polypeptide comprising an amino acid sequence that is 95% or more identical to SEQ ID NO:2 and includes an activity substantially similar to SEQ ID NO:2.

6) The polypeptide of claim 5, wherein activity comprises specifically binding a phosphorylated cyclin E polypeptide.

7) The polypeptide of claim 5, wherein activity comprises specifically binding an SCF complex.

8) The polypeptide of claim 5, wherein activity comprises enhancing a ubiquitination of a phosphorylated cyclin E polypeptide.

9) The polypeptide of claim 5, wherein activity comprises enhancing a cellular turnover of a cyclin E polypeptide.

10) The polypeptide of claim 5, wherein the amino acid sequence is 98% or more identical to SEQ ID NO:2.

11) The polypeptide of claim 5, wherein the amino acid sequence is 99% or more identical to SEQ ID NO:2.

12) A purified immunogenic composition comprising a polypeptide consisting essentially of 6 to 167 consecutive amino acid residues of SEQ ID NO:4.

13) The immunogenic composition of claim 12, wherein the polypeptide is combined with an immunogenicity enhancing agent.

14) The immunogenic composition of claim 12, wherein the composition further comprises an N-terminal cysteine operative linked to the polypeptide, and wherein the polypeptide consists essentially of SEQ ID NO:21.

15) The immunogenic composition of claim 12, wherein the polypeptide consists essentially of 10 to 20 consecutive amino acid residues of SEQ ID NO:4.

16) A composition comprising a purified polypeptide including an amino acid sequence set forth in SEQ ID NO:10.

17) The composition of claim 16, wherein the polypeptide consists essentially of the amino acid sequence set forth in SEQ ID NO:10.

18) The composition of claim 16, wherein the polypeptide comprises SEQ ID NO:6.

19) The composition of claim 16, wherein the polypeptide consists essentially of the amino acid sequence set forth in SEQ ID NO:6.

20) A purified polypeptide comprising an amino acid sequence that is 95% or more identical to SEQ ID NO:6 and includes an activity substantially similar to SEQ ID NO:6.

21) The polypeptide of claim 20, wherein activity comprises specifically binding a phosphorylated cyclin E polypeptide.

22) The polypeptide of claim 20, wherein activity comprises specifically binding an SCF complex.

23) The polypeptide of claim 20, wherein activity comprises enhancing a ubiquitination of a phosphorylated cyclin E polypeptide.

24) The polypeptide of claim 20, wherein activity comprises enhancing a cellular turnover of a cyclin E polypeptide.

25) A purified immunogenic composition comprising a polypeptide consisting essentially of 6 to 87 consecutive amino acid residues of SEQ ID NO:6.

26) The immunogenic composition of claim 25, wherein the polypeptide is combined with an immunogenicity enhancing agent.

27) The immunogenic composition of claim 25, wherein the polypeptide consists essentially of SEQ ID NO:23.

28) The immunogenic composition of claim 25, wherein the polypeptide consists essentially of 10 to 20 consecutive amino acid residues of SEQ ID NO:6.

29) A purified immunogenic composition comprising a polypeptide consisting essentially of 6 to 49 consecutive amino acid residues of SEQ ID NO:16.

30) The immunogenic composition of claim 29, wherein the polypeptide is combined with an immunogenicity enhancing agent.

31) The immunogenic composition of claim 29, wherein the composition further comprises an N-terminal cysteine operative linked to the polypeptide, and wherein the polypeptide consists essentially of SEQ ID NO:24.

32) The immunogenic composition of claim 29, wherein the polypeptide consists essentially of 10 to 20 consecutive amino acid residues of SEQ ID NO:16.

33) A purified immunogenic composition comprising a polypeptide consisting essentially of 6 to 20 consecutive amino acid residues of SEQ ID NO:14.

34) The immunogenic composition of claim 33, wherein the polypeptide is combined with an immunogenicity enhancing agent.

35) The immunogenic composition of claim 33, wherein the composition further comprises an N-terminal cysteine operative linked to the polypeptide, and wherein the polypeptide consists essentially of SEQ ID NO:26.

36) The immunogenic composition of claim 33, wherein the polypeptide consists essentially of 11 to 15 consecutive amino acid residues of SEQ ID NO:14.

37) An isolated antibody that specifically binds to an hCdc4 α-exon 1 polypeptide.

38) The antibody of claim 37, wherein the hCdc4 α-exon 1 polypeptide is set forth in SEQ ID NO:21.

39) The antibody of claim 37, wherein the hCdc4 α-exon 1 polypeptide is set forth in SEQ ID NO:4.

40) The antibody of claim 37, wherein the antibody does not specifically bind to a second polypeptide consisting essentially of one or more of the following:
a) SEQ ID NO:14;
b) an hCdc4 β-exon 1;
c) SEQ ID NO:10;
d) an hCdc4 γ-exon 1;
e) SEQ ID NO:16;
f) a β-hCdc4;
g) SEQ ID NO:6;
h) a γ-hCdc4; or
i) SEQ ID NO:18.

41) An isolated antibody that specifically binds to an hCdc4 β-exon 1 polypeptide.

42) The antibody of claim 41, wherein the hCdc4 β-exon 1 polypeptide is set forth in SEQ ID NO:23.

43) The antibody of claim 41, wherein the hCdc4 β-exon 1 polypeptide is set forth in SEQ ID NO:10.

44) The antibody of claim 41, wherein the antibody does not specifically bind to a second polypeptide consisting essentially of one or more of the following:
a) SEQ ID NO:14;
b) an hCdc4 α-exon 1;
c) SEQ ID NO:4;
d) an hCdc4 γ-exon 1;
e) SEQ ID NO:16;
f) an α-hCdc4;
g) SEQ ID NO:2;
h) a γ-hCdc4; or
i) SEQ ID NO:18.

45) An isolated antibody that specifically binds to an hCdc4 γ-exon 1 polypeptide.

46) The antibody of claim 45, wherein the hCdc4 γ-exon 1 polypeptide is set forth in SEQ ID NO:24.

47) The antibody of claim 45, wherein the hCdc4 γ-exon 1 polypeptide is set forth in SEQ ID NO:16.

48) The antibody of claim 45, wherein the antibody does not specifically bind to a second polypeptide consisting essentially of one or more of the following:
a) SEQ ID NO:14;
b) an hCdc4 α-exon 1
c) SEQ ID NO:4;
d) an hCdc4 β-exon 1;
e) SEQ ID NO:10;
f) an α-hCdc4;
g) SEQ ID NO:2;
h) a β-hCdc4; or
i) SEQ ID NO:6.

49) An isolated antibody that specifically binds to an amino acid sequence consisting essentially of SEQ ID NO:14, or an immunogenic fragment thereof.

50) The isolated antibody of claim 49, wherein the antibody specifically binds the immunogenic fragment KRKLDHGSEVRSFS (SEQ ID NO:26).

51) A purified polynucleotide comprising a nucleic acid sequence encoding a polypeptide including SEQ ID NO:4, or a complement of the nucleic acid sequence.

52) The polynucleotide of claim 51, wherein the polypeptide includes SEQ ID NO:2.

53) The polynucleotide of claim 51, wherein the polynucleotide further comprises a vector sequence operatively linked to the nucleic acid sequence.

54) An isolated host cell, comprising the polynucleotide of claim 53.

55) The polynucleotide of claim 51, wherein the nucleic acid sequence encodes an amino acid sequence consisting essentially of SEQ ID NO:4, or a complement of the nucleic acid sequence.

56) The polynucleotide of claim 51, wherein the nucleic acid sequence encodes an amino acid sequence consisting essentially of SEQ ID NO:2, or a complement of the nucleic acid sequence.

57) A purified polynucleotide that includes a sequence identify of 95% or more compared to the polynucleotide of claim 51.

58) An isolated host cell, comprising the polynucleotide of claim 57 operatively linked to a vector sequence and capable of expression of the polynucleotide.

59) A purified polynucleotide comprising a nucleic acid sequence encoding a polypeptide including SEQ ID NO:10, or a complement of the nucleic acid sequence.

60) The polynucleotide of claim 59, wherein the polypeptide includes SEQ ID NO:6.

61) The polynucleotide of claim 59, wherein the polynucleotide further comprises a vector sequence operatively linked to the nucleic acid sequence.

62) An isolated host cell, comprising: the polynucleotide of claim 61.

63) The polynucleotide of claim 59, wherein the nucleic acid sequence encodes an amino acid sequence consisting essentially of SEQ ID NO:10, or a complement of the nucleic acid sequence.

64) The polynucleotide of claim 59, wherein the nucleic acid sequence encodes an amino acid sequence consisting essentially of SEQ ID NO:6, or a complement of the nucleic acid sequence.

65) A purified polynucleotide comprising a mutant α-hCdc4 polynucleotide including one or more mutations in a codon 124, 367, 371, 465, 472, 479, or 658 of the polynucleotide, wherein a wild-type α-hCdc4 polynucleotide comprises a nucleic acid sequence set forth in SEQ ID NO:1.

66) A purified polypeptide expressed from the polynucleotide of claim 65, wherein a wild-type α-hCdc4 polypeptide comprises an amino acid sequence set forth in SEQ ID NO:2.

67) A purified polypeptide comprising a mutant α-hCdc4 polypeptide including a truncation mutation or a frame shift mutation in a C-terminal domain of the mutant α-hCdc4 polypeptide, wherein a wild-type α-hCdc4 polypeptide includes an amino acid sequence set forth in SEQ ID NO:2, and wherein a wild-type C. terminal domain includes a sequence set forth in SEQ ID NO:14.

68) The purified polypeptide of claim 67, wherein the truncation mutation or the frame shift mutation occurs at amino acid position 168 or greater relative to the wild-type α-hCdc4 polypeptide.

69) The purified polypeptide of claim 67, wherein the truncation mutation or the frame shift mutation occurs at amino acid position 284 or greater relative to the wild-type α-hCdc4 polypeptide.

70) The purified polypeptide of claim 67, wherein the truncation mutation or the frame shift mutation occurs at amino acid position 369 or greater relative to the wild-type α-hCdc4 polypeptide.

71) A purified polynucleotide comprising a nucleic acid sequence encoding the polypeptide of claim 67.

72) A purified polynucleotide comprising a mutant β-hCdc4 polynucleotide including a mutation in a codon 23 of the polynucleotide, wherein a wild-type β-hCdc4 polynucleotide comprises a nucleic acid sequence set forth in SEQ ID NO:5.

73) A polypeptide manufactured by expression of the polynucleotide of claim 72, wherein a wild-type α-hCdc4 polypeptide comprises an amino acid sequence set forth in SEQ ID NO:6.

74) A purified polypeptide comprising a mutant β-hCdc4 polypeptide including a truncation mutation or a frame shift mutation in a C-terminal domain of the mutant β-hCdc4 polypeptide, wherein a wild-type β-hCdc4 polypeptide includes an amino acid sequence set forth in SEQ ID NO:6, and wherein a wild-type C. terminal domain includes a sequence set forth in SEQ ID NO:14.

75) The purified polypeptide of claim 74, wherein the truncation mutation or the frame shift mutation occurs at amino acid position 88 or greater relative to the wild-type β-hCdc4 polypeptide.

76) The purified polypeptide of claim 74, wherein the truncation mutation or the frame shift mutation occurs at amino acid position 204 or greater relative to the wild-type α-hCdc4 polypeptide.

77) The purified polypeptide of claim 74, wherein the truncation mutation or the frame shift mutation occurs at amino acid position 289 or greater relative to the wild-type α-hCdc4 polypeptide.

78) A purified polynucleotide comprising a nucleic acid sequence encoding the polypeptide of claim 74.

79) A purified polynucleotide comprising a nucleic acid sequence encoding a cyclin E polypeptide including a T62 mutation, wherein a wild-type cyclin E polypeptide includes an amino acid sequence set forth in SEQ ID NO:31 or SEQ ID NO:32.

80) The polynucleotide of claim 79, wherein the mutation is a substitution mutation or a deletion mutation.

81) The polynucleotide of claim 79, wherein the mutation of the polypeptide is at amino acid position 62 of SEQ ID NO:32 or amino acid position 77 of SEQ ID NO:31.

82) A polypeptide manufactured by expression of the polynucleotide of claim 79.

83) The polynucleotide of claim 79, wherein the cyclin E polypeptide further includes a T380 mutation.

84) The polynucleotide of claim 79, wherein the T380 mutation of the polypeptide is at amino acid position 380 of SEQ ID NO:32 or amino acid position 395 of SEQ ID NO:31.

85) A polypeptide manufactured by expression of the polynucleotide of claim 79.

86) A method of detecting an α-hCdc4 expression product in a sample, comprising specifically determining that the sample includes an α-exon 1 sequence, wherein the α-exon 1 sequence is a ribonucleic acid sequence or an amino acid sequence.

87) The method of claim 86, wherein the α-exon 1 sequence is a ribonucleic acid sequence.

88) The method of claim 87, wherein the α-exon 1 sequence is set forth in SEQ ID NO:28.

89) The method of claim 86, wherein the α-exon 1 sequence is an amino acid sequence.

90) The method of claim 89, wherein the α-exon 1 sequence is set forth in SEQ ID NO:4.

91) A method of detecting a β-hCdc4 expression product in a sample, comprising specifically determining that the sample includes a β-exon 1 sequence, wherein the β-exon 1 sequence is a ribonucleic acid sequence or an amino acid sequence.

92) The method of claim 91, wherein the β-exon 1 sequence is a ribonucleic acid sequence.

93) The method of claim 92, wherein the β-exon 1 sequence is set forth in SEQ ID NO:29.

94) The method of claim 91, wherein the β-exon 1 sequence is an amino acid sequence.

95) The method of claim 94, wherein the β-exon 1 sequence is set forth in SEQ ID NO:10.

96) A method of detecting a γ-hCdc4 expression product in a sample, comprising specifically determining that the sample includes a γ-exon 1 sequence, wherein the γ-exon 1 sequence is a ribonucleic acid sequence or an amino acid sequence.

97) The method of claim 96, wherein the γ-exon 1 sequence is a ribonucleic acid sequence.

98) The method of claim 97, wherein the γ-exon 1 sequence is set forth in SEQ ID NO:30.

99) The method of claim 96, wherein the γ-exon 1 sequence is an amino acid sequence.

100) The method of claim 99, wherein the γ-exon 1 sequence is set forth in SEQ ID NO: 16.

101) A diagnostic system, comprising: a targeting agent that specifically binds to an α-exon 1 of an hCdc4 expression product and a detecting agent operably linked to the targeting agent.

102) The diagnostic system of claim 101, wherein the hCdc4 expression product is a ribonucleic acid sequence.

103) The diagnostic system of claim 102, wherein the detecting agent comprises a complementary polynucleotide operably linked to a detectable label.

104) The diagnostic system of claim 101, wherein the hCdc4 expression product is an amino acid sequence.

105) The diagnostic system of claim 104, wherein the targeting agent comprising an antibody.

106) A method of detecting a phosphorylated cyclin E in a sample, comprising:
a) contacting the sample with an antibody that specifically binds to the phosphorylated cyclin E; and
b) detecting a specific binding of the antibody, thereby detecting the phosphorylated cyclin E in the sample.

107) The method of claim 106, wherein the phosphorylated cyclin E includes a T62 phosphorylation, and wherein the antibody specifically binds to an epitope of the cyclin E including the T62 phosphorylation.

108) The method of claim 107, wherein the epitope is CSLIPTPDKEDDDRV (SEQ ID NO:36), wherein the threonine residue comprises the T62 phosphorylation.

109) The method of claim 106, wherein the phosphorylated cyclin E includes a T380 phosphorylation, and wherein the antibody specifically binds to an epitope of the cyclin E including the T380 phosphorylation.

110) The method of claim 109, wherein the epitope is SGLLTPPQSGKK (SEQ ID NO:38), wherein the threonine residue comprises the T62 phosphorylation.

111) The method of claim 106, wherein the antibody includes a binding site specific for a T62-phospho cyclin E and a second binding site specific for a T380-phospho cyclin E.

112) The method of claim 106, wherein the sample comprises human cells.

113) The method of claim 106, wherein the sample comprises a human bodily fluid.

114) The method of claim 106, further comprising identifying the sample to include an accumulation of phosphorylated cyclin E.

115) A purified polypeptide comprising an amino acid sequence CSLIPTPDKEDDDRV (SEQ ID NO:36), wherein the threonine residue at position six includes a phosphorylation.

116) The polypeptide of claim 115, consisting essentially of the amino acid sequence CSLIPTPDKEDDDRV (SEQ ID NO:36), wherein the threonine residue at position six includes a phosphorylation.

117) A purified polypeptide comprising an amino acid sequence CSLIPTPDKEDDDRV (SEQ ID NO:37), wherein the threonine residue at position six does not include a phosphorylation.

118) The polypeptide of claim 117, consisting essentially of the amino acid sequence CSLIPTPDKEDDDRV (SEQ ID NO:37), wherein the threonine residue at position six does not include a phosphorylation.

119) A purified polypeptide comprising an amino acid sequence SGLLTPPQSGKK (SEQ ID NO:38), wherein the threonine residue at position five includes a phosphorylation.

120) The polypeptide of claim 119, consisting essentially of the amino acid sequence SGLLTPPQSGKK (SEQ ID NO:38), wherein the threonine residue at position five includes a phosphorylation.

121) A purified polypeptide comprising an amino acid sequence SGLLTPPQSGKK (SEQ ID NO:39), wherein the threonine residue at position five does not include a phosphorylation.

122) The polypeptide of claim 121, consisting essentially of the amino acid sequence SGLLTPPQSGKK (SEQ ID NO:39), wherein the threonine residue at position five does not include a phosphorylation.

123) A purified polypeptide comprising an amino acid sequence CSGLLTPPQSGKK (SEQ ID NO:40), wherein the threonine residue at position six includes a phosphorylation.

124) The polypeptide of claim 123, consisting essentially of the amino acid sequence CSGLLTPPQSGKK (SEQ ID NO:40), wherein the threonine residue at position six includes a phosphorylation.

125) A purified polypeptide comprising an amino acid sequence CSGLLTPPQSGKK (SEQ ID NO:41), wherein the threonine residue at position six does not include a phosphorylation.

126) The polypeptide of claim 125, consisting essentially of the amino acid sequence CSGLLTPPQSGKK (SEQ ID NO:41), wherein the threonine residue at position six does not include a phosphorylation.

127) A purified antibody that selectively and specifically binds a phosphorylated cyclin E polypeptide.

128) A purified antibody that selectively and specifically binds a non-phosphorylated cyclin E.

129) A purified antibody that specifically binds to a phosphorylated cyclin E and a non-phosphorylated cyclin E.

130) The antibody of claim 129, wherein the antibody specifically binds to a cyclin E including a T62-phosphorylation.

131) The antibody of claim 129, wherein the antibody specifically binds to a cyclin E including a T380-phosphorylation.

132) A purified antibody that specifically binds to a cyclin E polypeptide including a phosphorylated T62 residue, wherein the polypeptide includes 6 or more amino acid residues.

133) The antibody of claim 132, wherein the polypeptide includes 10 to 30 amino acids.

134) The antibody of claim 132, wherein the polypeptide includes no more than 40 amino acids.

135) The antibody of claim 132, wherein the polypeptide consists essentially of CSLIPTPDKEDDDRV (SEQ ID NO:36), wherein the threonine residue at position six is phosphorylated.

136) The antibody of claim 132, wherein the antibody does not specifically bind CSLIPTPDKEDDDRV (SEQ ID NO:37), wherein the threonine residue at position six is not phosphorylated.

137) A purified antibody that specifically binds to a cyclin E polypeptide including a phosphorylated T380 residue, wherein the polypeptide includes 6 or more amino acid residues.

138) The antibody of claim 137, wherein the polypeptide includes 10 to 30 amino acids.

139) The antibody of claim 137, wherein the polypeptide includes no more than 40 amino acids.

140) The antibody of claim 137, wherein the antibody specifically binds SGLLTPPQSGKK (SEQ ID NO:38), wherein the threonine residue at position five is phosphorylated.

141) The antibody of claim 137, wherein the antibody does not specifically bind SGLLTPPQSGKK (SEQ ID NO:39), wherein the threonine residue at position five is not phosphorylated.

142) A method of detecting a tumor in a subject; comprising:
a) contacting a sample of the subject with an antibody that specifically binds to a phosphorylated cyclin E; and
b) detecting a specific binding of the antibody, thereby detecting the tumor in the subject.

143) The method of claim 142, wherein the antibody specifically binds to a T62-phospho cyclin E.

144) The method of claim 142, wherein the antibody specifically binds to a T380-phospho cyclin E.

145) The method of claim 142, wherein the tumor comprises an endometrial tumor.

146) The method of claim 142, wherein the tumor is a lung tumor, a tumor of the head, a neck tumor, a testicular tumor, or a breast tumor.

147) A method of determining a ratio of a phosphorylated cyclin E to a non-phosphorylated cyclin E in a sample, comprising:
a) measuring a specific binding of an anti-phospho-cyclin E antibody;
b) measuring a specific binding of an anti-cyclin E antibody; and
c) comparing the specific binding of the anti-phospho-cyclin E antibody to the specific binding of the anti-cyclin E antibody, thereby determining the ratio of the phosphorylated cyclin E to total cyclin E in the sample.

148) The method of claim 147, wherein the anti-phospho-cyclin E antibody specifically binds an amino acid sequence comprising CSLIPTPDKEDDDRV (SEQ ID NO:36), wherein the threonine residue is phosphorylated.

149) The method of claim 147, wherein the anti-phospho-cyclin E antibody specifically binds an amino acid sequence comprising SGLLTPPQSGKK (SEQ ID NO:38), wherein the threonine residue is phosphorylated.

150) The method of claim 147, wherein the anti-cyclin E antibody specifically binds an amino acid sequence comprising CSLIPTPDKEDDDRV (SEQ ID NO:37), wherein the threonine residue is not phosphorylated.

151) The method of claim 147, wherein the anti-cyclin E antibody specifically binds an amino acid sequence comprising SGLLTPPQSGKK (SEQ ID NO:39), wherein the threonine residue is not phosphorylated.

152) The method of claim 147, determining the ratio of the non-phosphorylated cyclin E to the phosphorylated cyclin E in the sample.

153) The method of claim 147, wherein the sample is a tissue or a fluid sample of a human.

154) The method of claim 153, wherein the human has a tumor.

155) The method of claim 153, wherein the human is suspected of having a tumor.
